SEQUENCE LISTING (42 sequences)

SEQ ID NO: 1 - DNA, 20 bp, Artificial Sequence
  Synthetic Partial nucleic acid sequence targeted by HBA 10
gggtttatgcttggggcgcg

SEQ ID NO: 2 - DNA, 23 bp, Artificial Sequence
  Synthetic Full nucleic acid sequence targeted by HBA 10
gggtttatgcttggggcgcgggg

SEQ ID NO: 3 - DNA, 20 bp, Artificial Sequence
  Synthetic Partial nucleic acid sequence targeted by HBA 12
gactcagagagaacccacca

SEQ ID NO: 4 - DNA, 23 bp, Artificial Sequence
  Synthetic Full nucleic acid sequence targeted by HBA 12
gactcagagagaacccaccatgg

SEQ ID NO: 5 - DNA, 20 bp, Artificial Sequence
  Synthetic Partial nucleic acid sequence targeted by HBA 14
tgggttctctctgagtctgt

SEQ ID NO: 6 - DNA, 23 bp, Artificial Sequence
  Synthetic Full nucleic acid sequence targeted by HBA 14
tgggttctctctgagtctgtggg

SEQ ID NO: 7 - DNA, 20 bp, Artificial Sequence
  Synthetic Partial nucleic acid sequence targeted by HBA 15 1
gggttctctctgagtctgtg

SEQ ID NO: 8 - DNA, 23 bp, Artificial Sequence
  Synthetic Full nucleic acid sequence targeted by HBA 15 1
gggttctctctgagtctgtgggg

SEQ ID NO: 9 - DNA, 20 bp, Artificial Sequence
  Synthetic Partial nucleic acid sequence targeted by HBA 16 1
gtcggcaggagacagcacca

SEQ ID NO: 10 - DNA, 23 bp, Artificial Sequence
  Synthetic Full nucleic acid sequence targeted by HBA 16 1
gtcggcaggagacagcaccatgg

SEQ ID NO: 11 - DNA, 20 bp, Artificial Sequence
  Synthetic Partial nucleic acid sequence targeted by HBA 17 G
gcaggagacagcaccatggt

SEQ ID NO: 12 - DNA, 23 bp, Artificial Sequence
  Synthetic Full nucleic acid sequence targeted by HBA 17 G
gcaggagacagcaccatggtggg

SEQ ID NO: 13 - DNA, 20 bp, Artificial Sequence
  Synthetic Partial nucleic acid sequence targeted by HBA 20 1
cataaaccctggcgcgctcg

SEQ ID NO: 14 - DNA, 23 bp, Artificial Sequence
  Synthetic Full nucleic acid sequence targeted by HBA 20 1
cataaaccctggcgcgctcgcgg

SEQ ID NO: 15 - DNA, 20 bp, Artificial Sequence
  Synthetic Partial nucleic acid sequence targeted by HBA 5 1
ttgaatgctccagccggttc

SEQ ID NO: 16 - DNA, 23 bp, Artificial Sequence
  Synthetic Full nucleic acid sequence targeted by HBA 5.1
ttgaatgctccagccggttccag

SEQ ID NO: 17 - DNA, 20 bp, Artificial Sequence
  Synthetic Partial nucleic acid sequence targeted by gRNA2
cgggaggcttcgcccaatcc

SEQ ID NO: 18 - DNA, 23 bp, Artificial Sequence
  Synthetic Full nucleic acid sequence target by gRNA2
cgggaggcttcgcccaatcctgg

SEQ ID NO: 19 - DNA, 20 bp, Artificial Sequence
  Synthetic Partial nucleic acid sequence targeted by gRNA19
cgggcgagcgagtgcgagcc

SEQ ID NO: 20 - DNA, 22 bp, Artificial Sequence
  Synthetic Full nucleic acid sequence targeted by gRNA19
cgggcgagcgagtgcgagccgg

SEQ ID NO: 21 - DNA, 20 bp, Artificial Sequence
  Synthetic Partial nucleic acid sequence targeted by gRNA11
gggaggcttcgcccaatcct

SEQ ID NO: 22 - DNA, 23 bp, Artificial Sequence
  Synthetic Full nucleic acid sequence targeted by gRNA11
gggaggcttcgcccaatcctggg

SEQ ID NO: 23 - DNA, 20 bp, Artificial Sequence
  Synthetic Partial nucleic acid sequence targeted by HBA INT1 72.1
caggccaccctcaaccgtcc

SEQ ID NO: 24 - DNA, 23 bp, Artificial Sequence
  Synthetic Full nucleic acid sequence targeted by HBA INT1 72.1
caggccaccctcaaccgtcctgg

SEQ ID NO: 25 - DNA, 20 bp, Artificial Sequence
  Synthetic Partial nucleic acid sequence targeted by HBA INT1 73.2
tccggggccaggacggttga

SEQ ID NO: 26 - DNA, 23 bp, Artificial Sequence
  Synthetic Full nucleic acid sequence targeted by HBA INT1 73.2
tccggggccaggacggttgaggg

SEQ ID NO: 27 - DNA, 20 bp, Artificial Sequence
  Synthetic Partial nucleic acid sequence targeted by HBA INT1 73b.1
gtccggggccaggacggttg

SEQ ID NO: 28 - DNA, 23 bp, Artificial Sequence
  Synthetic Full nucleic acid sequence targeted by HBA INT1 73b.1
gtccggggccaggacggttgagg

SEQ ID NO: 29 - DNA, 20 bp, Artificial Sequence
  Synthetic Partial nucleic acid sequence targeted by HBA INT2 13.2
ccctcgacccagatcgctcc

SEQ ID NO: 30 - DNA, 23 bp, Artificial Sequence
  Synthetic Full nucleic acid sequence targeted by HBA INT2 13.2
ccctcgacccagatcgctcccgg

SEQ ID NO: 31 - DNA, 20 bp, Artificial Sequence
  Synthetic Partial nucleic acid sequence targeted by HBA INT2 74.1
gcgtgatcctctgccctgag

SEQ ID NO: 32 - DNA, 23 bp, Artificial Sequence
  Synthetic Full nucleic acid sequence targeted by HBA INT2 74.1
gcgtgatcctctgccctgagagg

SEQ ID NO: 33 - DNA, 26 bp, Artificial Sequence
  Synthetic GAPDH transcript
cttcattgacctcaactacatggttt

SEQ ID NO: 34 - DNA, 22 bp, Artificial Sequence
  Synthetic GAPDH transcript
tgggatttccattgatgacaag

SEQ ID NO: 35 - DNA, 19 bp, Artificial Sequence
  Synthetic HBG1 transcript
cctgtcctctgcctctgcc

SEQ ID NO: 36 - DNA, 19 bp, Artificial Sequence
  Synthetic HBG2 transcript
ggattgccaaaacggtcac

SEQ ID NO: 37 - DNA, 22 bp, Artificial Sequence
  Synthetic HBB transcript
gcaaggtgaacgtggatgaagt

SEQ ID NO: 38 - DNA, 23 bp, Artificial Sequence
  Synthetic HBB transcript
taacagcatcaggagtggacaga

SEQ ID NO: 39 - DNA, 21 bp, Artificial Sequence
  Synthetic HBA transcript
cggtcaacttcaagctcctaa

SEQ ID NO: 40 - DNA, 20 bp, Artificial Sequence
  Synthetic HBA transcript
acagaagccaggaacttgtc

SEQ ID NO: 41 - DNA, 19 bp, Artificial Sequence
  Synthetic HBD transcript
caagggcactttttctcag

SEQ ID NO: 42 - DNA, 19 bp, Artificial Sequence
  Synthetic HBD transcript
aattccttgccaaagttgc

(19) United States
(12) Patent Application Publication
(10) Pub. No.: US 2022/0184137 A1
(43) Pub. Date: Jun. 16, 2022

(54) Correction of Beta-Thalassemia Phenotype by Genetically Engineered Hematopoietic Stem Cell

(71) Applicants: INSERM (Institut National de la Santé et de la Recherche Médicale), Paris (FR); Université d'Evry val d'Essonne, Evry (FR); Genethon, Evry (FR)

(72) Inventors: Mario AMENDOLA, Evry (FR); Giulia PAVANI, Evry (FR)

(21) Appl. No.: 17/603,220

(22) PCT Filed: Apr. 10, 2020
(86) PCT No.: PCT/EP2020/060309

§ 371 Date: Oct. 12, 2021

(30) Foreign Application Priority Data
Apr. 12, 2019 (EP) 19305484.8

(51) Int. Cl. A61K 35/28; C12N 5/0789; C12N 15/113; C07K 14/805

(52) U.S. Cl. CPC A61K 35/28; C12N 5/0647; C12N 2750/14143; C07K 14/805; C12N 2510/00; C12N 15/113

(57) ABSTRACT

The present invention relates to a genetically modified hematopoietic stem cell (HSC) comprising, in at least one α-globin gene comprised in the genome thereof, at least one transgene encoding a functional β-like globin protein, the said transgene being placed under the control of the endogenous promoter of the said at least one α-globin gene.

### FIELD OF THE INVENTION

The present invention relates to the field of therapeutic treatment by genome engineering.

In particular, the invention relates to a genetically modified hematopoietic stem cell (HSC) and to its use as a medicament, more particularly to its use in the treatment of β-hemoglobinopathies, in particular for use in the treatment of β-thalassemia.

### BACKGROUND OF THE INVENTION

Hemoglobinopathies are a kind of genetic defect that results in abnormal structure of one of the globin chains of the hemoglobin molecule and are among the most common inherited diseases around the world.

Adult hemoglobin consists of two pairs of globin subunits (α2/β2), whose production is strictly regulated to ensure balanced expression in erythroid cells.

β-thalassemia and sickle cell disease (SCD) are the world's two most widely disseminated hereditary hemoglobinopathies. Originally, β-thalassemia emerged in the Mediterranean, Middle Eastern, and Asian regions, and SCD in central Africa. However, subsequent population migration caused these two diseases to become global and as such constitute a growing health problem in many countries.

β-thalassemias are a group of inherited blood diseases caused by a genetic deficiency in the synthesis of the β-chains of hemoglobin, due to mutations in the HBB gene on chromosome 11. Mutated alleles are called (3+ when partial function is conserved (either the protein has a reduced function, or it functions normally but is produced in reduced quantity) or β0, when no functioning protein is produced.

The severity of the disease depends on the nature of the mutation and on the presence of mutations in one or both alleles. (3 thalassemia major (Mediterranean anemia or Cooley anemia) is caused by a β0/β0 genotype. No functional (3 chains are produced, and thus no hemoglobin A can be assembled. This is the most severe form of β-thalassemia. β thalassemia intermedia is caused by a β+/β0 or β+/β+ genotype. In this form, some hemoglobin A is produced. β thalassemia minor is caused by a β/β0 or β/β+ genotype. Only one of the two β globin alleles contains a mutation, so β chain production is not severely compromised and patients may be relatively asymptomatic.

Several mutations in the β-globin gene cluster, such as those that occur in β-thalassemic individuals, can alter a/β-globin chain balance leading to accumulation and precipitation of free α-globin chains. There are two genes for α-globin (HBA1 and HBA2), and therefore four α-globin genes in diploid cells (which can be represented as αα/αα). The coding sequences of HBA1 and HBA2 are identical.

These highly toxic α-globin chain aggregates damage cell membranes causing hemolysis and ineffective erythropoiesis.

These disorders result in variable outcomes ranging from severe anemia to clinically asymptomatic individuals. Thousands of infants with β-thalassemia are born each year.

Despite remarkable improvements in medical care for patients with β-hemoglobinopathies, only one definitive treatment option exists: allogeneic hematopoietic stem cell (HSC) transplantation. The development of gene therapy for β-hemoglobinopathies has been justified by (1) the limited availability of human leukocyte antigen (HLA)-identical donors, (2) the narrow window of application of HSC transplantation to the youngest patients, and (3) recent advances in HSC-based gene therapy.

In particular, in order to reduce this imbalance between the production of β- and α-globin chains, most of the current clinical approaches aim to increase the β- and β-like globin chains. For example, Dever et al. (Nature 2016; 539(7629): 384-389) achieved homologous recombination (HR) at the β-globin gene in HSCs by combining Cas9 ribonucleoproteins and rAAV6 HR donor delivery.

Alternatively, globin balance has been described as being improved by reducing the production of α-globin chains. Metamananda et al. (Nature communications 8, 424 (2017)) accordingly proposed the genome editing of an α-globin enhancer in primary human HSC as a treatment for β-thalassemia. Papadaki and Vassilopoulos (Thalassemia reports 2013; 3(s1):e40) developed vectors targeting the α-globin transcript using the shRNA technology.

In particular, Papadaki and Vassilopoulos developed foamy virus (FV) vectors for the production of β-globin in addition to the above-mentioned vectors targeting the α-globin transcript. However, this method happens to be toxic for the cells, and can lead to the risk of insertional mutagenesis or oncogene transactivation by enhancer proximity effect, which has been previously reported to have tumorigenic potential.

Moreover, none of the approaches currently available, and in particular none of the approaches mentioned above, is completely satisfactory, more particularly when it comes to treating individuals, who suffer from severe forms of β-thalassemia, such as β-thalassemia major (also represented here-after using the terms β0 thalassemic patients; β⁰ cells; or β0-thal, which concern cells or patients having no residual β-globin chain expression).

Therefore, there remains a need to develop novel gene therapy platforms to treat hemoglobinopathies, and in particular β-thalassemia, which are more efficient and safer than those known in the art.

In particular, there is a need for a method allowing to restore, in vitro or ex vivo, the physiological level of production of hemoglobin in a cell, and in particular in a blood cell, in a safe and efficient manner.

### SUMMARY OF THE INVENTION

The Applicant managed to identify a method allowing to respond to the needs mentioned above.

A first object of the present invention accordingly relates to a genetically modified hematopoietic stem cell (HSC) comprising, in at least one α-globin gene comprised in the genome thereof, at least one transgene encoding a functional β-like globin protein, in particular a functional β-globin protein, the said transgene being placed under the control of an endogenous sequence allowing the transcription and/or enhancing the transcription, in particular the endogenous promoter, of the said at least one α-globin gene.

In a particular embodiment, the at least one α-globin gene comprising the at least one transgene encoding a functional β-like globin protein does not express a gene sequence encoding a functional α-globin protein.

In an embodiment, the at least one transgene is comprised in at least two α-globin genes, preferably in no more than two α-globin genes. In particular, the at least two α-globin genes can be on different chromosomes.

In another particular embodiment, the at least one transgene encoding a functional β-like globin protein is comprised in the 5′ untranslated region (5′UTR) and/or in the proximal promoter and/or in the second intron (IVS2) of the said at least one α-globin gene, preferably is comprised in the 5′ untranslated region (5′ UTR) or in the proximal promoter or in the second intron (IVS2) of the said at least one α-globin gene.

In a further embodiment, the at least one α-globin gene is selected from the group consisting of the α 1 globin gene and the α 2 globin gene.

The inventors developed a unique, easy and fast, as well as accessible, genome editing method for restoring, in vitro or ex vivo, the physiological level of production of hemoglobin in a cell, in particular in a red blood cell, originating from a genetically modified HSC according to the invention, and more particularly for treating hemoglobinopathies. This method is moreover safe and non-toxic.

To the inventors' knowledge, such an advantageous combination therapy allowing to simultaneously inactivate at least one α-globin gene and increase the expression of at least one β-like globin gene has never been described in the prior art to efficiently treat hemoglobinopathies, and in particular β-thalassemia.

Another object of the present invention relates to a blood cell originating from a genetically modified HSC according to the invention.

A further object of the present invention relates to a pharmaceutical composition comprising at least one genetically modified HSC and/or at least one blood cell according to the invention, in a pharmaceutically acceptable medium.

Another object of the present invention relates to a method for the ex vivo or in vitro preparation of a HSC according to the invention, comprising at least the steps of:

(i) providing to the said stem cell a site-directed genetic engineering system by:
(a) providing to the said stem cell (1) at least one guide nucleic acid binding to a selected target site or (2) at least one guide peptide-containing endonuclease binding to a selected target site, the said target site being located in an endogenous α-globin gene comprised in the genome of the said HSC;
(b) when the at least one guide nucleic acid has been provided at step (a)(1), further providing to the said stem cell at least one endonuclease devoid of target site specificity; and
(c) further providing to the said stem cell at least one transgene that encodes a functional β-like globin protein, in particular a β-globin protein;

and

(ii) culturing the stem cell obtained at step (i) such that the said at least one transgene encoding a functional β-like globin protein is introduced at the said selected target site in the genome of the said HSC and placed under the control of the endogenous sequence allowing the transcription and/or enhancing the transcription of the said at least one α-globin gene, in particular under the control of the endogenous promoter of the said at least one α-globin gene.

In particular, said method can be such that it comprises the steps of:

(i) providing to the said stem cell a site-directed genetic engineering system by:
(a) providing to the said stem cell at least one guide nucleic acid binding to a selected target site, the said target site being located in an α-globin gene comprised in the genome of the said HSC;
(b) further providing to the said stem cell at least one endonuclease devoid of target site specificity; and
(c) further providing to the said stem cell at least one transgene that encodes a functional β-like globin protein;

and

(ii) culturing the stem cell obtained at the end of step (i) such that the said at least one transgene is introduced at the said selected target site in the genome of the said HSC.

In particular, said method can be such that it comprises the steps of:

(i) providing to the said stem cell a site-directed genetic engineering system by:
(a) providing to the said stem cell at least one guide nucleic acid binding to a selected target site, the said target site being located in an endogenous α-globin gene comprised in the genome of the said hematopoietic stem cell;
(b) further providing to the said stem cell at least one Clustered regularly interspaced short palindromic repeats (CRISPR) associated nuclease; and
(c) further providing to the said stem cell at least one transgene that encodes a functional β-like globin protein;

and

(ii) culturing the stem cell obtained at the end of step (i) such that the said at least one transgene is introduced at the said selected target site in the genome of the said hematopoietic stem cell.

In a particular embodiment, the at least one endonuclease devoid of target site specificity is a Clustered regularly interspaced short palindromic repeats (CRISPR) associated nuclease, in particular the CRISPR associated protein 9 (Cas9).

Another object of the present invention relates to a HSC, a blood cell or a pharmaceutical composition according to the invention for its use as a medicament.

A further object of the present invention relates to a HSC, a blood cell, or a pharmaceutical composition according to the invention, for use in the treatment of β-hemoglobinopathies, in particular for use in the treatment of β-thalassemia.

An aim of the present invention is to provide an in vitro or ex vivo method for genetically editing a HSC having a β-hemoglobinopathic phenotype so that it is able to differentiate into a cell, in particular a blood cell, and more particularly a red blood cell, having a restored physiological level of production of hemoglobin.

Another object of the present invention thus relates to an in vitro or ex vivo method for restoring the physiological level of production of hemoglobin in a red blood cell originating from a genetically modified HSC according to the invention. The present invention relates to an in vitro or ex vivo method for restoring the physiological level of production of hemoglobin in a cell, in particular in a blood cell, more particularly in a red blood cell, comprising at least the steps of:

(i) providing to the cell a site-directed genetic engineering system by:
(a) providing to the said cell (1) at least one guide nucleic acid binding to a selected target site or (2) at least one guide peptide-containing endonuclease binding to a selected target site, the said target site being located in an endogenous α-globin gene comprised in the genome of the said cell;
(b) when the at least one guide nucleic acid has been provided at step (a)(1), further providing to the said cell at least one endonuclease devoid of target site specificity; and
(c) further providing to the said cell at least one transgene that encodes a functional β-like globin protein, in particular a functional β-globin protein;

(ii) culturing the cell obtained at step (i) such that the said at least one transgene encoding a functional β-like globin protein is introduced at the said selected target site in the genome of the said cell and placed under the control of the endogenous sequence allowing the transcription and/or enhancing the transcription of the said at least one α-globin gene, in particular under the control of the endogenous promoter of the said at least one α-globin gene; and

(iii) culturing the cell obtained at step (ii) such that the at least one transgene encoding a functional β-like globin protein that has been introduced in the genome of the cell is transcribed by the endogenous transcription system of the cell.

A further object of the present invention relates to a kit for restoring in vitro or ex vivo the physiological level of production of hemoglobin in a cell, in particular in a blood cell, more particularly a red blood cell, comprising:

(i) at least one guide nucleic acid binding to a selected target site or at least one guide peptide-containing endonuclease binding to a selected target site, the said target site being located in an endogenous α-globin gene comprised in the genome of a HSC;

(ii) when the at least one guide nucleic acid is provided as part (i), further at least one endonuclease devoid of target site specificity; and

(iii) at least one transgene encoding a functional β-like globin protein, in particular a functional β-globin protein.

### DESCRIPTION OF THE FIGURES

FIG. 1 Design and validation of gRNAs in K562 erythroleukemia cell line

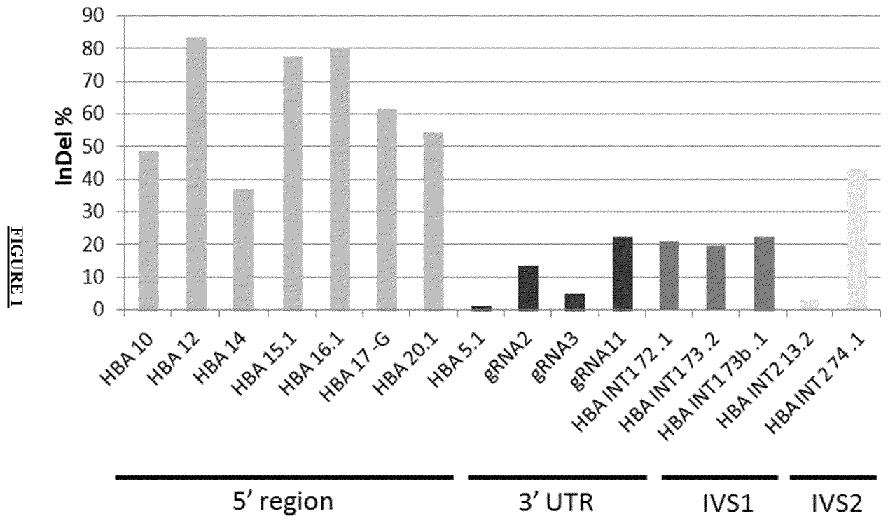

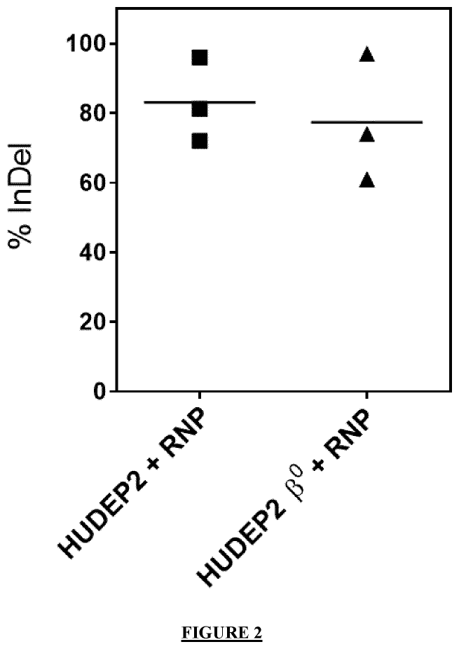

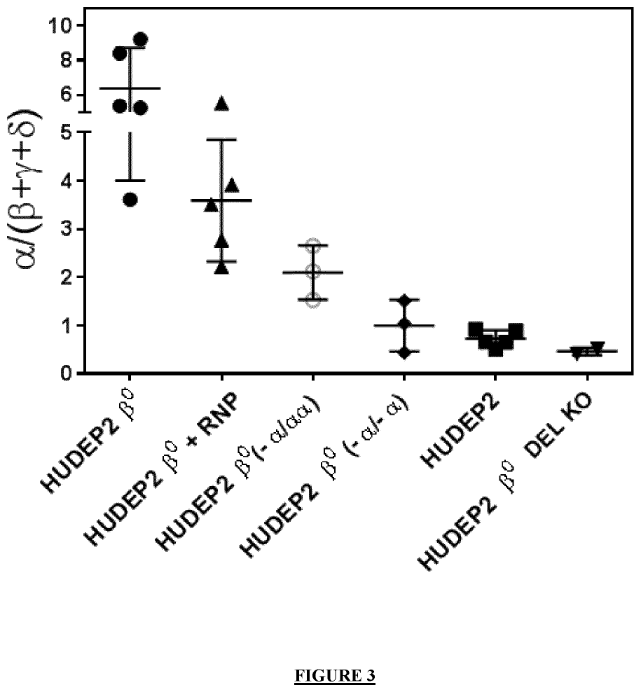

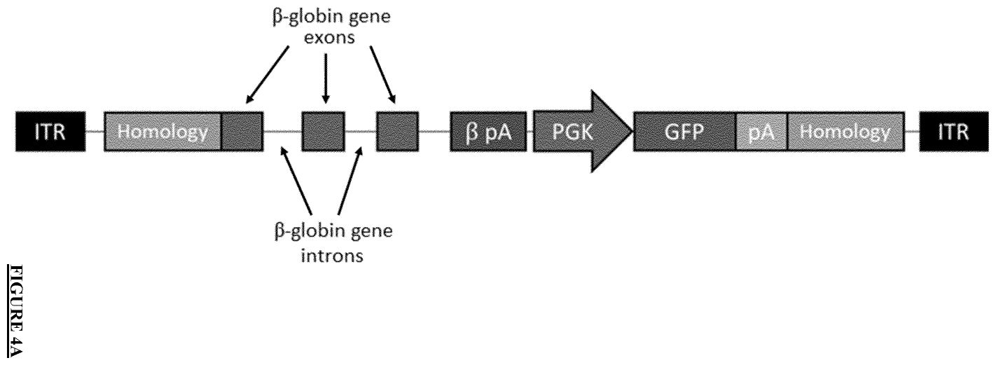

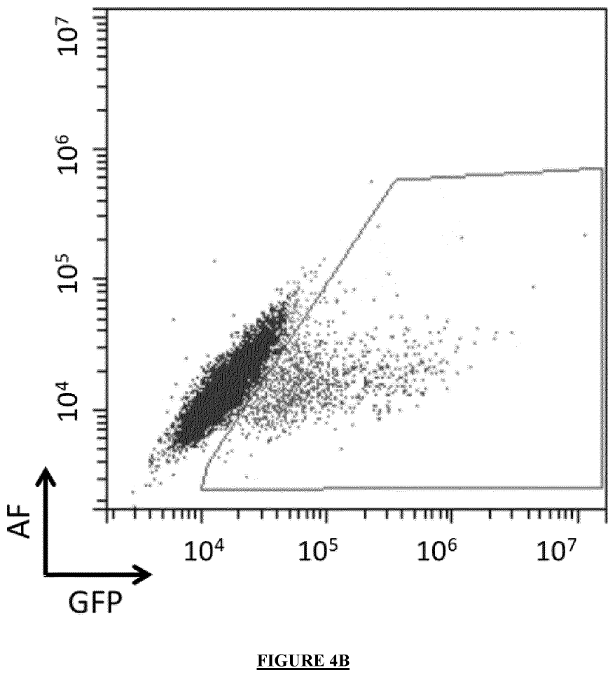

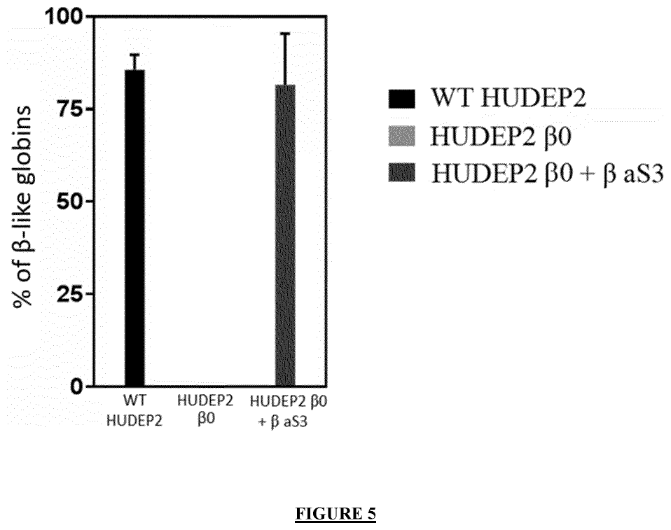

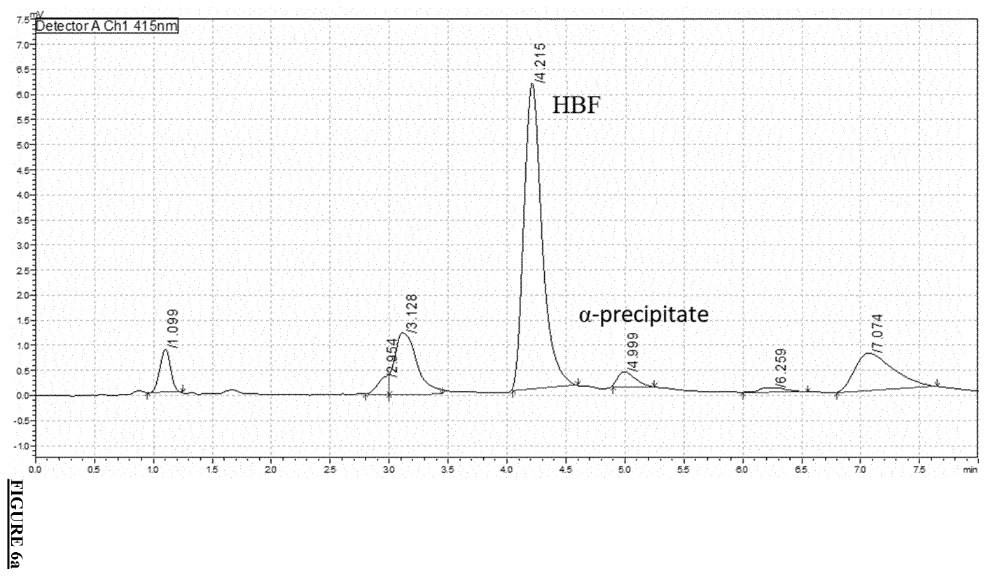

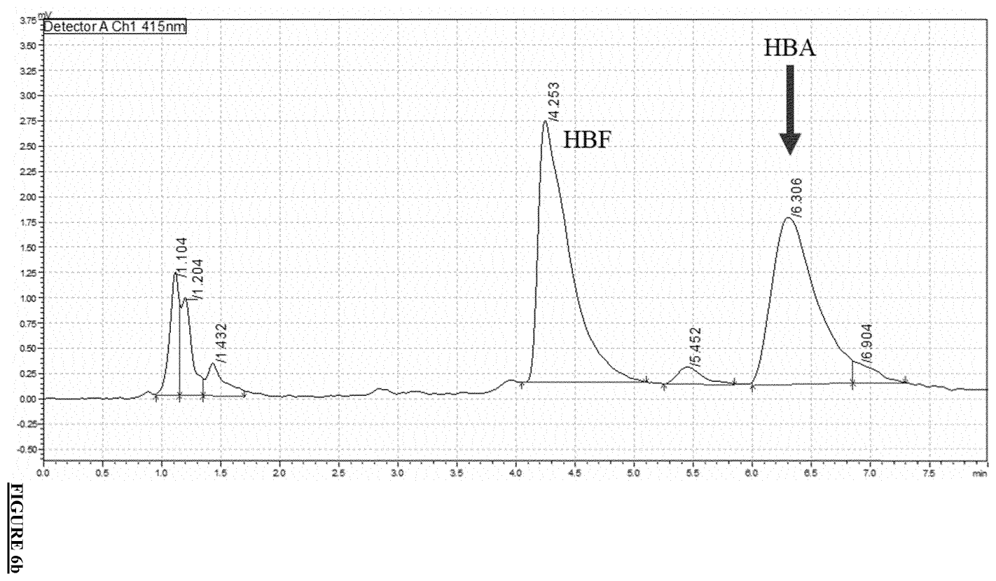

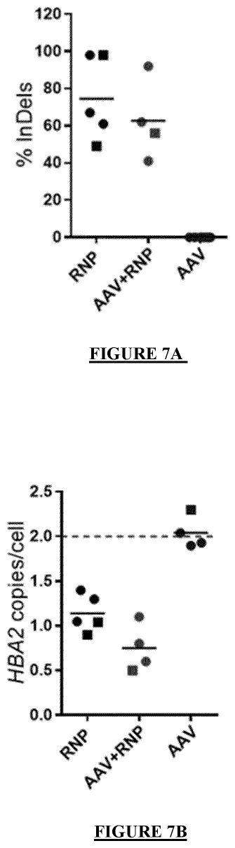

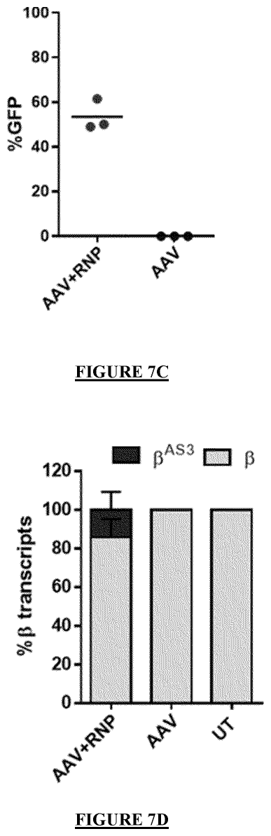

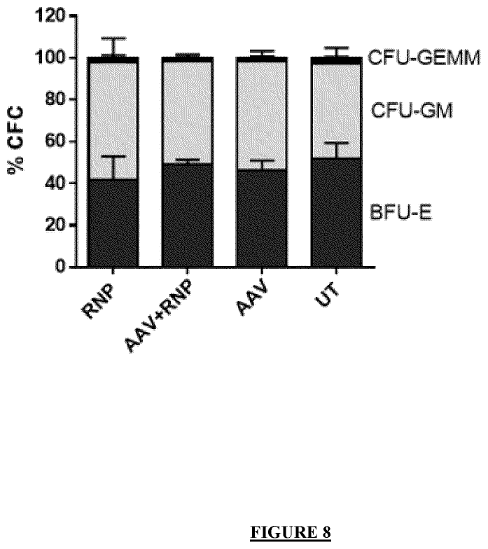

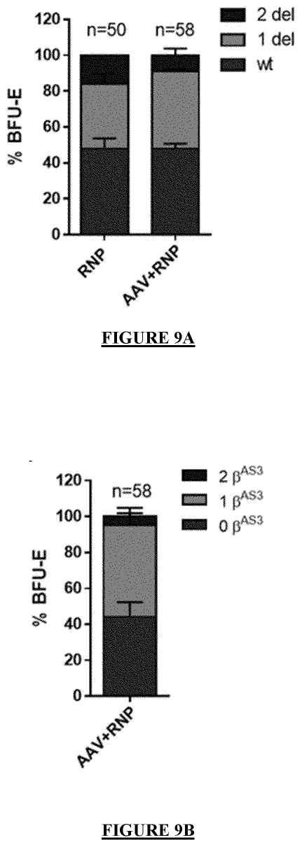

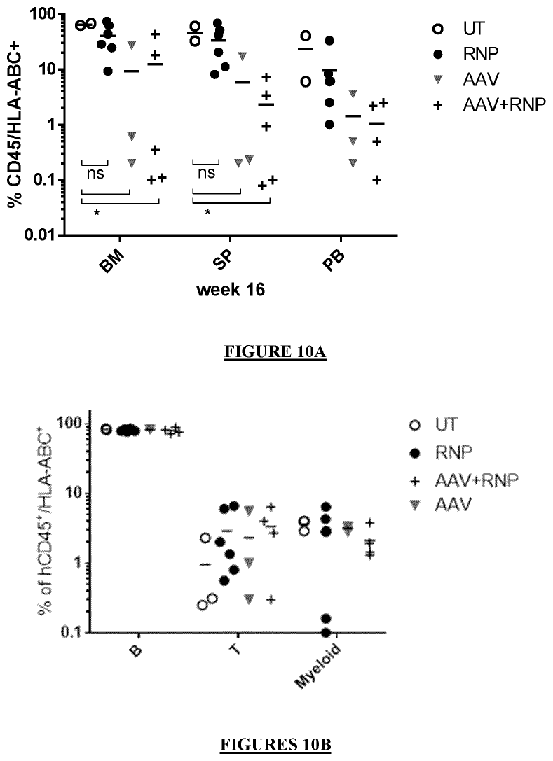

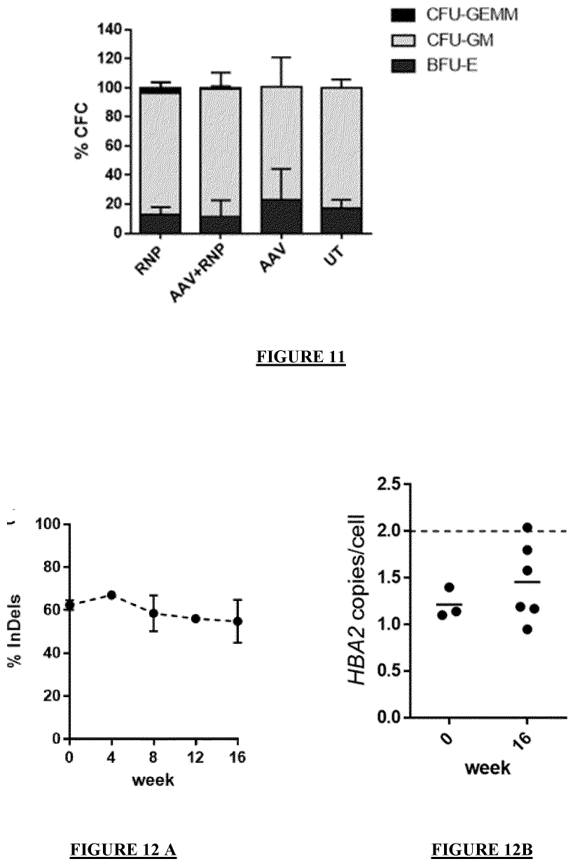

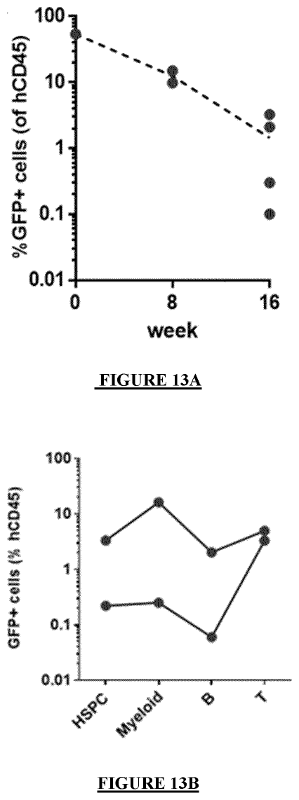

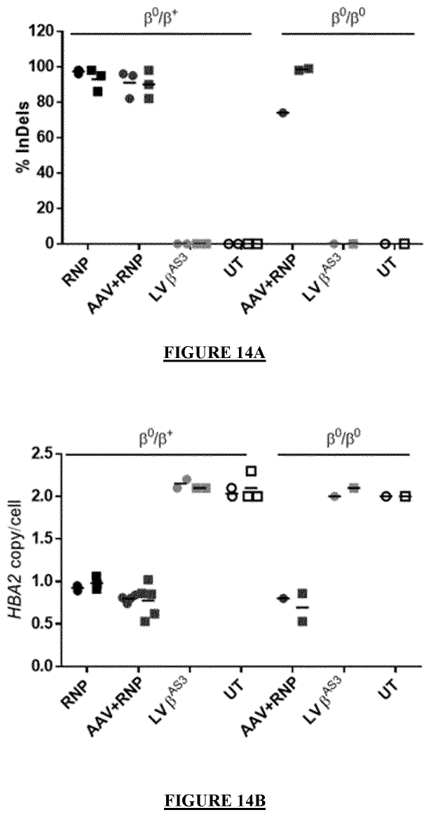

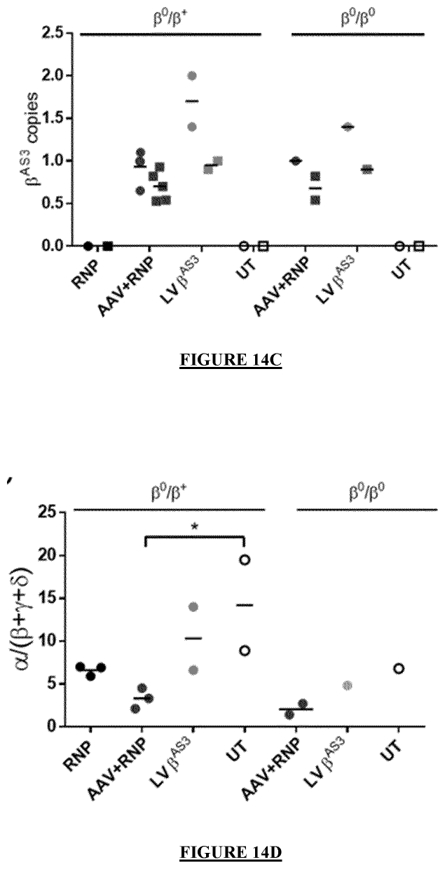

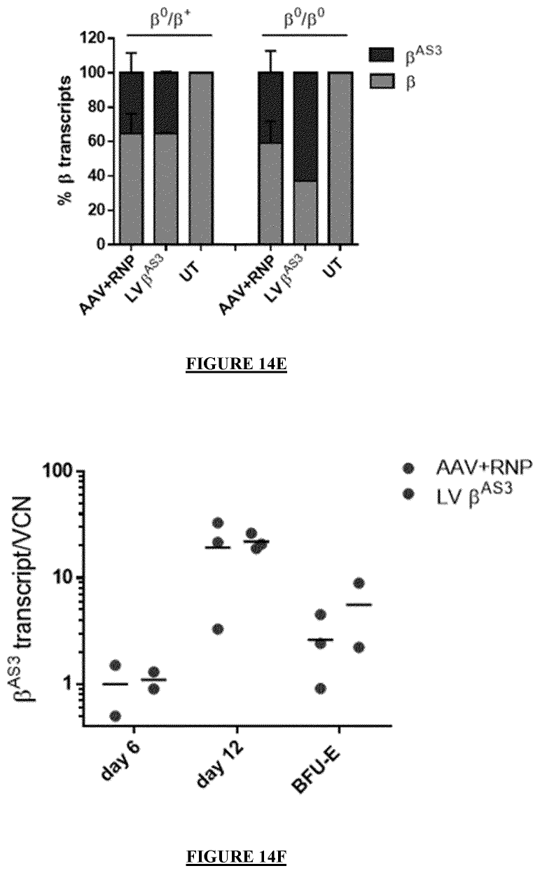

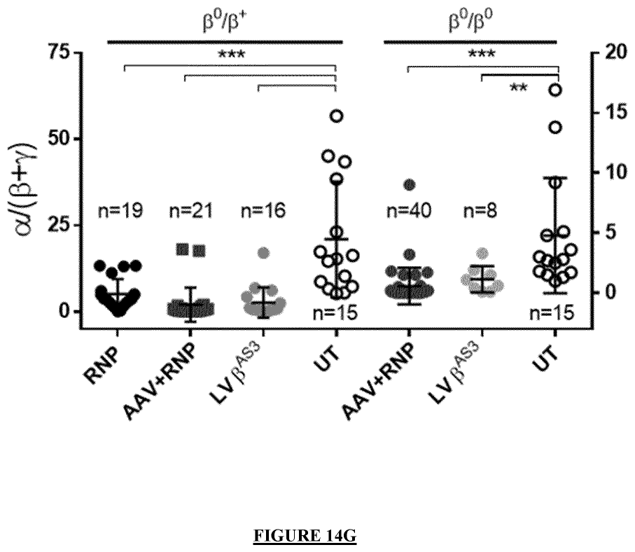

Plasmids expressing different gRNA for HBA were transfected in K562 cells stabling expressing SpCas9 and their DNA cutting activity was measured with TIDE software (Tracking of InDels by Decomposition—Brinkman et al. Nucleic Acids Res. 2014. 42(22):e168); www.tide.calculator.nk). The activity is represented in FIG. 1 as InDel %, i.e. percentage of modified alleles, for each of the 16 gRNA tested independently either in the 5′ region of the gene as further defined (in particular the 5′ UTR of HBA), in the 3′ untranslated region (3′ UTR) of the gene as further defined, in intron 1 (IVS1—for Intervening Sequence 1) or in intron 2 (IVS2) of the said gene. The closer to 100% a InDel %, the more efficient the gRNA tested.

Abscissa: the region targeted by the tested gRNA: 5′ region, IVS1 or IVS2; each bar represents a different gRNA.

Ordinate: the InDel %.

FIG. 2: Preparation of α-globin deleted clones of human erythroid progenitor cells (HUDEP2 β0) simulating a β-thalassemic phenotype

Human erythroid progenitor cell line (HUDEP2) cells or human erythroid progenitor cell line cells having no residual β-globin chain expression (HUDEP2 β0) were nucleofected with Cas9:gRNA ribonucleoprotein targeting HBA genes and nuclease activity was measured on the basis of the percentage of Insertions and Deletions (InDel), calculated by using the software TIDE (Tracking of InDels by Decomposition) available on the website www.tide.calculator.nk.

The activity is represented as InDel %, i.e. a percentage of modified alleles, for each gRNA tested.

The closer to 100% a InDel %, the more efficient the gRNA tested.

Abscissa: from left to right: results obtained with HUDEP2 cells, results obtained with HUDEP2 β0 cells.

Ordinate: the InDel %.

FIG. 3: globin balance in the cells generated in example 2

HUDEP2 (WT control), HUDEP2 β0 or Cas9 edited HUDEP2 β0 (HUDEP2 β0+RNP) cells were differentiated and globin RNA was measured. Different clones of edited HUDEP2 β0 cells with one (−α/αα) or 2 α-globin deletions (−α/−α) were also selected and differentiated. Globin ratio is close to 1 in differentiated HUDEP2, >1 in HUDEP2 β0 and <1 in “HUDEP2 β0 DEL KO”, a control clone knock-out for both (3 and a globins.

Abscissa: from left to right: results obtained with HUDEP2 β0 cells, with HUDEP2 β0+RNP cells (mixture of all different types of edited HUDEP2 β0 in terms of α-globin deletions) with HUDEP2 β0 (−α/αα) cells, with HUDEP2 β0 (−α/−α) cells, with WT HUDEP2 cells (HUDEP2) and with HUDEP2 β0 DEL KO cells.

Ordinate: ratio of a to β-like mRNA (beta, gamma and delta globins).

FIGS. 4a and 4b: Integration of a β-globin transgene in thalassemic HUDEP2 β0 cells

FIG. 4A: AAV vector structure containing the following elements, from left to right: ITR, Inverted Terminal Repeat, Homology: ˜250 bp DNA sequence homologous to the genomic sequence where the nuclease cutting is occurring; β-globin gene: boxes represent exons, while lines represent introns; (3 pA: β-globin polyadenylation sequence; PGK: human phosphoglycerate kinase promoter; GFP: green fluorescent protein; pA: polyadenylation sequence; particular the β-globin gene.

FIG. 4B: Targeted integration of the AAV in the 5′UTR of α-globin gene of HUDEP2 β0. At 14 days cells were subject to flow cytometry to analyze GFP expression coming from integrated AAV.

Abscissa: GFP signal intensity (Fluorescence intensity) Ordinate: Auto fluorescence channel (AF)

FIG. 5: HPLC analysis of β-globin expression in the genetically engineered cells

This Figure represents the percentage of β-like globins (ordinate) expressed in wild-type HUDEP2 cells (WT HUDEP2) compared to the corresponding percentage expressed in HUDEP2 β0 cells (HUDEP2 β0), and in HUDEP2 β0 cells into which a β globin transgene has been incorporated (HUDEP2 β0+β aS3) (abscissa, from left to right).

FIGS. 6a and 6b: HPLC analysis of hemoglobin subunits and tetramers in the genetically engineered cells

In FIG. 6a, peaks corresponding in particular to the α-precipitates formed in HUDEP2 β0 cells are identified and annotated using a loading control (Bio-rad, hemoglobin quality control). As expected, no HbA tetramers are detected in HUDEP2 β0 cells.

In FIG. 6b, peaks corresponding in particular to the tetramers (HbA) formed expressed in HUDEP2 β0 cells into which a (3 globin transgene has been incorporated are identified and annotated using a loading control. It can be seen that there is no α-precipitates detected.

Abscissa: Time (minutes)

Ordinate: Electrical signal (mV)

FIGS. 7A, 7B, 7C and 7D: HBA2 deletion and β^{AS3} integration efficiency in HSPCs FIG. 7A represents the editing efficiencies in HSPCs at day 12 of erythroid differentiation in erythroid liquid culture (•) or in BFU-E (burst-forming unit-erythroid, ▪). Lines represent mean.

Abscissa (from left to right): group of HSPCs administered only with ribonucleoprotein complexes (RNP), group of HSPCs administered with AAV as defined in the examples and ribonucleoprotein complexes (AAV+RNP) and group of HSPCs only administered with AAV as defined in the examples (AAV).

Ordinate: the InDel %.

FIG. 7B represents the number of HBA2 gene copies per HSPC in erythroid liquid culture (•) or in BFU-E (burst-forming unit-erythroid, ▪). Full lines represent mean; dotted line indicates the number of expected HBA2 alleles in untreated HSPCs.

Abscissa (from left to right): group of HSPCs administered only with ribonucleoprotein complexes (RNP), group of HSPCs administered with AAV as defined in the examples and ribonucleoprotein complexes (AAV+RNP) and group of HSPCs only administered with AAV as defined in the examples (AAV).

Ordinate: the number of HBA2 copies per cell.

FIG. 7C represents the KI efficiency, characterized by the % GFP, in edited HSPCs in erythroid liquid culture (•). Line represents mean.

Abscissa (from left to right): group of HSPCs administered with AAV as defined in the examples and ribonucleoprotein complexes (AAV+RNP) and group of HSPCs only administered with AAV as defined in the examples (AAV).

Ordinate: the % of GFP.

FIG. 7D represents the relative abundance of endogenous β and KI-β^{AS3} mRNA at day 12 of erythroid liquid culture (n=6).

Abscissa (from left to right): group of HSPCs administered with AAV as defined in the examples and ribonucleoprotein complexes (AAV+RNP), group of HSPCs only administered with AAV as defined in the examples (AAV) and group of untreated control (UT).

Ordinate: the % of β transcripts.

FIG. 8: Ex vivo pluripotency of edited HSPCs

FIG. 8 represents the colony formation unit (CFU) frequency in edited HSPCs. In each column, from top to bottom: CFU-GEMM (granulocyte, erythroid, macrophage, megakaryocyte); BFU-E (burst-forming unit-erythroid); CFU-GM (granulocyte, macrophage).

Bars represent mean±SD (n=3-5).

Abscissa (from left to right): group of HSPCs administered only with ribonucleoprotein complexes (RNP), group of HSPCs administered with AAV as defined in the examples and ribonucleoprotein complexes (AAV+RNP), group of HSPCs only administered with AAV as defined in the examples (AAV) and group of untreated control (UT).

Ordinate: the % of CFC.

FIGS. 9A and 9B: HBA2 deletion and β^{AS3} integration efficiency in BFU-E

FIG. 9A represents the HBA2 gene deletion in single BFU-E: from bottom to top of each column: wild type (wt), one HBA2 deletion (1del) and two HBA2 deletions (2 del). Bars represent mean.

Abscissa (from left to right): group of HSPCs administered only with ribonucleoprotein complexes (RNP) and group of HSPCs administered with AAV as defined in the examples and ribonucleoprotein complexes_(AAV+RNP).

Ordinate: % of BFU-E.

FIG. 9B represents the β^{AS3} integration pattern in single BFU-E. Bars represent mean±SD (colonies derived from 2 independent experiments): from bottom to the top of the column: no integration (0), monoallelic (1) and biallelic (2) KI.

Ordinate: % of BFU-E.

FIGS. 10A and 10B: Engraftment and differentiation efficiency of edited HSPCs transplanted in immunodeficient NOD/SCID/γ (NSG) mice

FIG. 10A represents the percentage of human CD45+/HLA-ABC+ cells in hematopoietic organs of mice at week 16.

Black lines indicate mean.

Abscissa (from left to right): BM=bone marrow; SP=spleen; PB=peripheral blood. For each hematopoietic organ, from left to right: group of untreated control (UT) (∘), group of HSPCs administered only with ribonucleoprotein complexes (RNP) (•), group of HSPCs only administered with AAV as defined in the examples (AAV) (▾) and group of HSPCs administered with AAV as defined in the examples and ribonucleoprotein complexes (AAV+RNP) (+).

Ordinate: % of CD45+/HLA-ABC+.

FIG. 10B represents the immunophenotyping of human xenografts in bone marrow of NSG mice. Each dot represents one animal, lines indicate mean.

Abscissa (from left to right): human B CD19+(B), human T CD3+(T) and human myeloid CD33+(My) cells. For each hematopoietic organ, from left to right: group of untreated control (UT) (∘), group of HSPCs administered only with ribonucleoprotein complexes (RNP) (•), group of HSPCs only administered with AAV as defined in the examples (AAV) (▾) and group of HSPCs administered with AAV as defined in the examples and ribonucleoprotein complexes (AAV+RNP) (+).

Ordinate: % of CD45+/HLA-ABC+.

FIG. 11: Differentiation confirmation by CFC assay of isolated human CD34+ cells from bone marrow of engrafted mice from the previous examples

FIG. 11 represents the colony formation unit (CFU) frequency in bone marrow derived CD34. From bottom to top in each column: BFU-E (burst-forming unit-erythroid); CFU-GM (granulocyte, macrophage); and CFU-GEMM (granulocyte, erythroid, macrophage, megakaryocyte).

Bars represent mean±SD (n=2-4).

Abscissa (from left to right): group of HSPCs administered only with ribonucleoprotein complexes (RNP), group of HSPCs administered with AAV as defined in the examples and ribonucleoprotein complexes (AAV+RNP), group of HSPCs only administered with AAV as defined in the examples (AAV) and group of untreated control (UT).

Ordinate: % of CFC units.

FIGS. 12A and 12B: Comparison of the percentage of InDels (FIG. 12A), as well as the extent of HBA2 deletion (FIG. 12B), between ex vivo and in vivo RNP treated HSPCs

FIG. 12A represents the InDel efficiency in the peripheral blood of RNP engrafted mice at different timepoints (mean±SD; n=2-4).

Abscissa: time (in weeks).

Ordinate: the InDels %.

FIG. 12B represents HBA2 copies in RNP treated HSPCs at day 0 (injection) and in BM of engrafted mice at w16. Full lines indicate mean, dotted line indicates the number of expected HBA2 alleles in untreated HSPCs.

Abscissa: time (in weeks).

Ordinate: the number of HBA2 copies per cell.

FIGS. 13A and 13B: Control of the presence of β^{AS3} KI HSPC (GFP positive) at different time points (FIG. 13A) and in different lineages (FIG. 13B)

FIG. 13A represents the GFP positive cells in the peripheral blood of transplanted mice over time. GFP is expressed as percentage of CD45+ cells; line indicates mean.

Abscissa: time (in weeks).

Ordinate: the % GFP positive cells (GFP+ cells).

FIG. 13B represents the GFP positive cells in HSPCs (CD34), myeloid (CD33), B (CD19) and T (CD3) cells in BM of transplanted mice. Each line represents one animal.

Abscissa (from left to right): HSPC, Myeloid cells, B cells and T cells.

Ordinate: the % GFP positive cells (GFP+ cells).

FIGS. 14A, 14B, 14C, 14D, 14E, 14F and 14G: Genome editing of the α-globin locus ameliorates globin balance in thalassemic HSPCs

FIG. 14A represents the InDels quantification in erythroid liquid culture (•) or in BFU-E (burst-forming unit-erythroid, ▪). Black lines represent means.

Abscissa (from left to right):

β0/β+ (from left to right: group of HSPCs administered only with ribonucleoprotein complexes (RNP), group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT); and

β0/β0 (from left to right: group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT).

Ordinate: the InDels %.

FIG. 14B represents the number of HBA2 gene copies per cell in erythroid liquid culture (•) or in BFU-E (burst-forming unit-erythroid, ▪). Black lines represent mean.

Abscissa (from left to right):

β0/β+ (from left to right: group of HSPCs administered only with ribonucleoprotein complexes (RNP), group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT); and

β0/β0 (from left to right: group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT).

Ordinate: number of HBA2 copies/cell.

FIG. 14C represents β^{AS3} integration in edited thalassemic HSPCs in erythroid liquid culture (•) or in BFU-E (burst-forming unit-erythroid, ▪). Black lines represent mean.

Abscissa (from left to right):

β0/β+ (from left to right: group of HSPCs administered only with ribonucleoprotein complexes (RNP), group of HSPCs administered with AAV as defined in the examples and ribonucleoprotein complexes (AAV+RNP), group of HSPCs administered only with the lentiviral vector encoding the βAS3 gene under the control of the erythroid specific 3-globin enhancer/promoter (LV β^{AS3}) and group of untreated control (UT); and

β0/β0 (from left to right: group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT).

Ordinate: number of β^{AS3} copies.

FIG. 14D represents the α/β-like globin mRNA ratios in edited thalassemic HSPCs in erythroid liquid culture at day 12, black lines indicate mean.

Abscissa (from left to right):

β0/β+ (from left to right: group of HSPCs administered only with ribonucleoprotein complexes (RNP), group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT)); and

β0/β0 (from left to right: group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT)).

Ordinate: α/(β+γ+δ) ratio.

FIG. 14E represents relative abundance of endogenous (3 and KI-β^{AS3} mRNA at day 12 of erythroid liquid culture (mean±SD, n=3).

Abscissa (from left to right):

β0/β+ (from left to right: group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT); and

β0/β0 (from left to right: group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT)).

FIG. 14F represents β^{AS3} RNA expression during erythroid differentiation in (3+ erythroblasts. Values are normalized per vector copy number (VCN) and then a β^{AS3}/GAPDH is performed (GAPDH=normalizer). Black lines indicate mean (n=2-3).

Abscissa (from left to right):

at day 6 (left: group of HSPCs administered with AAV as defined in the examples and ribonucleoprotein complexes (AAV+RNP), right: group of HSPCs administered only with the lentiviral vector encoding the βAS3 gene under the control of the erythroid specific β-globin enhancer/promoter (LV β^{AS3}));

at day 12 (left: group of HSPCs administered with AAV as defined in the examples and ribonucleoprotein complexes (AAV+RNP), right: group of HSPCs administered only with the lentiviral vector encoding the βAS3 gene under the control of the erythroid specific β-globin enhancer/promoter (LV β^{AS3})); and

in BFU-E (left: group of HSPCs administered with AAV as defined in the examples and ribonucleoprotein complexes (AAV+RNP), right: group of HSPCs administered only with the lentiviral vector encoding the βAS3 gene under the control of the erythroid specific β-globin enhancer/promoter (LV β^{AS3})).

Ordinate: β^{AS3} transcript/VCN.

FIG. 14G represents α/β-like globin mRNA ratios in BFU-E derived from edited thalassemic HSPCs. β0/β+ colonies (n=71) are plotted on the left axis, β0/β0 (n=63) on the right axis. Each dot represents a single colony. Black lines indicate mean±SD (***, p<0.001; **, p<0.01; ANOVA, Tukey's test).

Abscissa (from left to right):

β0/β+ (from left to right: group of HSPCs administered only with ribonucleoprotein complexes (RNP), group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT)); and

β0/β0 (from left to right: group of HSPCs administered with AAV as defined in the examples and ribonucleoprotein complexes (AAV+RNP), group of HSPCs administered only with the lentiviral vector encoding the βAS3 gene under the control of the erythroid specific β-globin enhancer/promoter (LV β^{AS3}) and group of untreated control (UT)).

### DETAILED DESCRIPTION OF THE INVENTION

The inventors managed to generate genetically modified HSCs that, when differentiated towards the erythroid lineage, are able to produce one or more β-like globin proteins encoded by at least one transgene encoding a functional β-like globin protein. In particular, the inventors managed to correct the hemoglobin production and the α-globin and β-like globin balance, and in particular the α-globin and β-globin balance in cells presenting a β-thalassemic, and in particular a severe β-thalassemic profil, using an easy to implement, safe, efficient and non-toxic method.

The inventors were also able to edit healthy donor HSPCs and demonstrated that they maintained long term repopulation capacity and multipotency in transplanted mice.

Finally, to assess the clinical potential of the approach, the inventors edited β-thalassemic HSPCs and achieved a clear improvement of the α/β globin balance in HSPC derived erythroblasts.

As shown herein, the said β-like globin protein(s) is (are) expressed at a high level in cells initially presenting a β-thalassemic profil, which allows obtaining therapeutic levels of the β-like globin protein as well as a therapeutic level of hemoglobin tetramers.

The inventors also managed to significantly reduce the production of toxic α-globin precipitates in these cells.

The genetically modified HSCs as described herein advantageously provide a controlled and restored expression of the β-like globin protein.

Another important advantage provided by the present invention is that it does not negatively affect the overall globin expression level of α-globin chains.

The use of an ex vivo or in vitro engineered HSC as described herein by transgene targeted integration in the safe α-globin gene advantageously minimizes the risk of insertional mutagenesis and oncogene transactivation associated with the use of semi-random integrating vectors and the risk of gene transactivation, as no exogenous promoter/enhancer elements are required for transgene expression and inserted in the genome.

Moreover, administration of the HSC as described herein to an individual in need thereof will allow for a long-term correction of β-hemoglobinopathies, in particular β-thalassemia, considered in the present text by restoring or providing additional function to these stem cells.

This method is highly advantageous to the individual in need thereof, to which the engineered HSC are administered for treatment of their pathology, as most of the current treatments for the hemoglobinopathies consist in frequent injections of healthy and immune compatible red blood cells, which is demanding and not curative on the long term. Such treatment is only symptomatic and not curative. Accordingly, the patients must undergo repeated administration of these proteins for the rest of their life.

Treatment based on the administration of a HSC as described herein results on the contrary in a limited number of repeated administrations or even in a one-time curative treatment with two major benefits: it will significantly improve the quality of life of the patients and their family and it will reduce the economic cost and burden on the national health system related to the treatment of these most often life-long diseases.

### Genetically Modified Hematopoietic Stem Cell (HSC)

As indicated above, the present invention firstly relates to a genetically modified HSC comprising, in at least one α-globin gene comprised in the genome thereof, at least one transgene encoding a functional β-like globin protein, and in particular a functional β-globin protein, the said transgene being placed under the control of an endogenous sequence allowing the transcription and/or enhancing the transcription of the said at least one α-globin gene.

HSC are pluripotent stem cells capable of self-renewal and are characterized by their ability to give rise under permissive conditions to all cell types of the hematopoietic system. HSC are not totipotent cells, i.e. they are not capable of developing into a complete organism.

In a particular embodiment, a HSC according to the invention is derived from an embryonic stem cell, in particular from a human embryonic stem cell, and is thus an embryonic hematopoietic stem cell.

Embryonic stem cells (ESCs) are stem cells derived from the undifferentiated inner mass cells of an embryo and capable of self-renewal. Under permissive conditions, these pluripotent stem cells are capable of differentiating in any one of the more than 220 cell types in the adult body. ESC are not totipotent cells, i.e. they are not capable of developing into a complete organism. ESC can for example be obtained according to the method indicated in Young Chung et al. (Cell Stem Cell 2, 2008 February 7; 2(2):113-7).

In another particular embodiment, a HSC according to the invention is an induced pluripotent stem cell, more particularly a human induced pluripotent stem cell (hiPSCs). Thus, according to a particular embodiment, HSC as described herein are hematopoietic induced pluripotent stem cells.

hiPSCs are genetically reprogrammed adult cells that exhibit a pluripotent stem cell-like state similar to ESC. They are artificially generated stem cells that are not known to exist in the human body but show qualities similar to those of ESC. Generating such cells is well known in the art as discussed in Ying WANG et al. (https://doi.org/10.1101/050021) as well as in Lapillonne H. et al. (Haematologica. 2010; 95(10)) and in J. DIAS et al. (Stem Cells Dev. 2011; 20(9): 1639-1647).

“Self renewal” refers to the ability of a cell to divide and generate at least one daughter cell with the identical (e.g., self-renewing) characteristics of the parent cell. The second daughter cell may commit to a particular differentiation pathway. For example, a self-renewing HSC can divide and form one daughter stem cell and another daughter cell committed to differentiation in the myeloid or lymphoid pathway. Self-renewal provides a continual source of undifferentiated stem cells for replenishment of the hematopoietic system.

The marker phenotypes useful for identifying HSCs will be those commonly known in the art. For human HSCs, the cell marker phenotypes preferably include any combination of CD34⁺CD38^{low/-} Cd49f⁺ CD59⁺ CD90⁺ CD45RA⁻ Thy1⁺ C-kit⁺ lin⁻ (Notta F, Science. 333(6039):218-21 (2011)). For mouse HSCs, the cell marker phenotypes can illustratively be any combination of CD34^{low/-} Sca-1⁺ C-kit⁺ and lin⁻ CD150⁺ CD48⁻ CD90.1.Thy1^{+/low} Flk2/flt3⁻ and CD117⁺, (see, e.g., Frascoli et al. (J. Vis. Exp. 2012 Jul. 8; (65). Pii:3736.).

Stem cells as described herein are preferably purified. The same applies for blood cells as defined herein.

Many methods for purifying HSC are known in the art, as illustrated for example in EP1687411.

As used herein, “purified HSC” or “purified blood cells” means that the recited cells make up at least 50% of the cells in a purified sample; more preferably at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more of the cells in a purified sample.

The cells' selection and/or purification can include both positive and negative selection methods to obtain a substantially pure population of cells.

In one aspect, fluorescence activated cell sorting (FACS), also referred to as flow cytometry, can be used to sort and analyze the different cell populations. Cells having the cellular markers specific for HSC or a progenitor cell population are tagged with an antibody, or typically a mixture of antibodies, that binds the cellular markers. Each antibody directed to a different marker is conjugated to a detectable molecule, particularly a fluorescent dye that can be distinguished from other fluorescent dyes coupled to other antibodies. A stream of stained cells is passed through a light source that excites the fluorochrome and the emission spectrum from the cells detects the presence of a particular labelled antibody. By concurrent detection of different fluorochromes, cells displaying different sets of cell markers are identified and isolated from other cells in the population. Other FACS parameters, including, by way of example and not limitation, side scatter (SSC), forward scatter (FSC), and vital dye staining (e.g., with propidium iodide) allow selection of cells based on size and viability. FACS sorting and analysis of HSC and progenitor cells is described in, among others, Akashi, K. et al., Nature 404(6774):193-197 (2000)).

In another aspect, immunomagnetic labelling can be used to sort the different cell population. This method is based on the attachment of small magnetizable particles to cells via antibodies or lectins. When the mixed population of cells is placed in a magnetic field, the cells that have beads attached will be attracted by the magnet and may thus be separated from the unlabeled cells.

In a preferred embodiment, the HSCs that will be genetically modified according to the invention present a β-hemoglobinopathy phenotype, i.e. present a diminished expression of β-like globin compared to a healthy corresponding cell. In particular, the HSC cells that will be genetically modified according to the invention present a β-thalassemia or sickle-cell disease phenotype.

In a particular embodiment, a modified HSC as described herein is a mammalian cell and in particular a human cell.

In a particular embodiment, the initial population of HSC and/or blood cells may be autologous.

“Autologous” refers to deriving from or originating in the same patient or individual. An “autologous transplant” refers to the harvesting and reinfusion or transplant of a subject's own cells or organs. Exclusive or supplemental use of autologous cells can eliminate or reduce many adverse effects of administration of the cells back to the host, particular graft versus host reaction.

In this case, the HSC were collected from the said individual, genetically modified ex vivo or in vitro according to a method as described herein and administered to the same individual.

In particular, the initial population of HSC and/or blood cell originates from an individual suffering from a β-hemoglobinopathy, in particular from β-thalassemia or sickle-cell disease, more particularly from β-thalassemia, and even more particularly from a severe form of β-thalassemia, such as β-thalassemia major.

In a particular embodiment, the initial population of HSC and/or blood cells may be derived from an allogeneic donor or from a plurality of allogeneic donors. The donors may be related or unrelated to each other, and in the transplant setting, related or unrelated to the recipient (or individual).

The stem cells to be modified as described herein may accordingly be exogenous to the individual in need of therapy.

Other embodiments of the invention utilizing endogenous HSC involve the mobilization of the said stem cells from one anatomical niche of the individual to systemic circulation, or into another specific anatomical niche. Such mobilization is well known in the art and may for example be caused by administration of factors capable of stimulating stem cell exodus from compartments such as the bone marrow.

Where applicable, stem cells and progenitor cells may be mobilized from the bone marrow into the peripheral blood by prior administration of cytokines or drugs to the subject (see, e.g., Domingues et al. (Int. J. Hematol. 2017 February; 105(2): 141-152)). Cytokines and chemokines capable of inducing mobilization include, by way of example and not limitation, granulocyte colony stimulating factor (G-CSF), granulocyte macrophage colony stimulating factor (GM-CSF), erythropoietin (Kiessinger, A. et al., Exp. Hematol. 23:609-612 (1995)), stem cell factor (SCF), AMD3100 (AnorMed, Vancouver, Canada), interleukin-8 (IL-8), and variants of these factors (e.g., pegfilgastrim, darbopoietin).

Cells prepared by a method as described herein may be resuspended in a pharmaceutically acceptable carrier and used directly or may be subjected to processing by various cell purification techniques available to the skilled artisan, such as FACS sorting, magnetic affinity separation, and immunoaffinity columns.

As already specified herein, the HSC and blood cells as described herein are genetically modified in at least one of its globin gene, in particular in at least one of its endogenous globin gene.

Globin genes are organized in clusters in the genome of HSC and blood cells, these clusters being called the α- and β-like human globin gene clusters.

The α-like human globin gene cluster comprises the zeta (ζ), pseudozeta (ψζ), mu (μ), pseudoα-1 (ψα1), pseudoα-2 (ψα2), α 2 (α2), α 1 (α1) and theta (θ) globin genes and is located on the chromosome 16.

The β-like human globin gene cluster comprises the epsilon (ε), gamma-G (G γ), gamma-A (A γ), delta (δ) and β (β) globin genes and is located on the chromosome 11.

Accordingly, a β-like globin gene according to the invention refers to a gene selected from the group consisting of epsilon-globin (c), gamma-G-globin (G γ), gamma-A-globin (A γ), delta-globin (δ) and β-globin (β) genes. In particular, a β-like globin gene according to the invention is a β-globin gene.

According to a particular embodiment, before modification, an HSC as discussed above is not capable of differentiating into an erythroblast that can express a functional β-globin protein (representing a β0 phenotype), i.e. the HSC is capable of differentiating into an erythroblast, but the erythroblast will not be able to express a functional β-globin protein (representing a β0 phenotype).

According to another embodiment, before modification, an HSC as discussed above is capable of differentiating into an erythroblast, but the erythroblast partially expresses a functional β-globin protein. That is, before modification, the HSC is capable of differentiating into an erythroblast, and said erythroblast either expresses a β-globin protein that has reduced function, or it expresses a β-globin protein that functions normally but is produced in a reduced quantity (representing a β+ phenotype).

As mentioned above, the at least one transgene encoding a functional β-like globin protein is comprised in at least one α-globin gene in the genome of HSC and blood cells.

As commonly defined in the art, a gene locus is a fixed position of a gene on a chromosome.

In the present text, when the terms “a cell” without any more indication is mentioned, it applies to both a HSC and to a blood cell as described herein.

According to an embodiment, the at least one α-globin gene is selected from the group consisting of the α1-globin gene and the α2-globin gene.

The coding region of each α-globin gene is interrupted at two positions by stretches of noncoding DNA called intervening sequences (IVSs) or introns.

An α-globin gene is thus constituted, from its 5′ end to its 3′ end, of:
a proximal promoter region;
a 5′ untranslated region (5′ UTR);
at least 2 exons, in particular 3 exons;
at least 1 intron, in particular two introns; and/or
a 3′ untranslated region (3′ UTR).

Accordingly, in a cell as described herein, the at least one transgene encoding a functional β-like globin protein comprised in the at least one α-globin gene of a cell as described herein can be comprised in the 5′ region, in an intron and/or in the 3′UTR of the said α-globin gene, in particular in the proximal promoter region, in the 5′ UTR and/or in an intron of the said α-globin gene.

Preferably, the at least one transgene encoding a functional β-like globin protein comprised in the at least one α-globin gene of a cell as described herein is comprised in the 5′ region and/or in an intron of the said α-globin gene, in particular in the 5′ UTR and/or in the proximal promoter and/or in an intron of the said α-globin gene, more particularly in the 5′ UTR or in the proximal promoter or in an intron of the said α-globin gene.

In a particular embodiment, the at least one transgene encoding a functional β-like globin protein comprised in the at least one α-globin gene of a cell as described herein is comprised in the 5′ region and/or in an intron, in particular in the 5′ region and/or in the second intron (IVS2) of the said at least one α-globin gene.

The 5′ region according to the invention is the region upstream the translation initiation codon of the considered α-globin gene. It comprises the 5′UTR sequence of the gene and the proximal promoter.

In particular, the 5′ region of an α-globin gene according to the invention corresponds to the 500 nucleotides sequence directly upstream said translation initiation codon of the α-globin gene, preferably the 400 nucleotides, more preferably the 300 nucleotides and more particularly the 250 nucleotides directly upstream said translation initiation codon of the α-globin gene.

In a particular embodiment, the at least one transgene encoding a functional β-like globin protein comprised in the at least one α-globin gene of a cell as described herein is comprised in the 5′ region, in the 3′ untranslated region (3′ UTR) and/or in an intron of the said at least one α-globin gene, in particular in the 5′ region and/or in the second intron (IVS2) of the said at least one α-globin gene.

In a particular embodiment, the at least one transgene encoding a functional β-like globin protein comprised in the at least one α-globin gene of a cell as described herein is comprised in the 5′ untranslated region (5′UTR) and/or in the proximal promoter and/or in the second intron (IVS2) of the said at least one α-globin gene, preferably is comprised in the 5′ untranslated region (5′ UTR) or in the proximal promoter or in the second intron (IVS2) of the said at least one α-globin gene.

In a particular embodiment, the 5′ region of an α-globin gene according to the invention corresponds to the proximal promoter of the considered α-globin gene.

In another embodiment, the 5′ region of an α-globin gene according to the invention corresponds to the 5′UTR (5′ untranslated region) of the considered α-globin gene.

All the following positions are based on UCSC Genome Browser on Human December 2013 GRCh38/hg38 Assembly.

In particular, when considering the HBA1 (hemoglobin subunit α 1) human gene, the 5′ region preferably corresponds to 5′UTR of this gene. This 5′UTR corresponds to the position chr16:176, 651-176, 716; 66 nt; RefSeq: NM_000558.4.

In particular, when considering the HBA2 (hemoglobin subunit α 2) human gene, the 5′ region preferably corresponds to the 5′UTR of this gene. This 5′UTR corresponds to the position chr16:172, 847-172, 912; 66 nt; RefSeq: NM_000517.4.

More particularly, the at least one transgene encoding a functional β-like globin protein comprised in the at least one α-globin gene of a cell as described herein can be comprised in the 5′ region, in the first intron (IVS1) and/or in the second intron (IVS2) of the said α-globin gene, preferably in the 5′ UTR and/or in the proximal promoter and/or in the second intron (IVS2) of the said α-globin gene, more preferably in the 5′ UTR and/or in the proximal promoter or in the second intron (IVS2) of the said α-globin gene.

When considering the HBA1 (hemoglobin subunit α 1) human gene, the first intron (IVS1) can correspond to the 117 nucleotides comprised between the first exon and the second exon of this gene. This region corresponds to the position chr16:176, 812-176, 928; 117 nt; RefSeq: NM_000558.4.

When considering the HBA2 (hemoglobin subunit α 2) human gene, the first intron (IVS1) can correspond to the 117 nucleotides comprised between the first exon and the second exon of this gene. This region corresponds to the position chr16:173, 008-173, 124; 117 nt; RefSeq: NM_000517.4.

When considering the HBA1 (hemoglobin subunit α 1) human gene, the second intron (IVS2) can correspond to the 149 nucleotides comprised between the second exon and the third exon of this gene. This region corresponds to the position chr16:177, 134-177, 282; 149 nt; RefSeq: NM_000558.4.

When considering the HBA2 (hemoglobin subunit α 2) human gene, the second intron (IVS2) can correspond to the 142 nucleotides comprised between the second exon and the third exon of this gene. This region corresponds to the position chr16:173, 330-173, 471; 142 nt; RefSeq: NM_000517.4.

The inventors indeed unexpectedly determined, as illustrated in the enclosed examples, that very good InDel percentages, defined further in the present text, as well as high transgene (GFP) expression are obtained when genome editing of a β-like globin gene occurs in selected locations of the α-globin gene.

In a particular embodiment, the at least one α-globin gene comprising the at least one transgene encoding a functional β-like globin protein does not express a gene sequence encoding a functional α-globin protein.

In another particular embodiment, the at least one transgene is comprised in at least two α-globin genes, preferably in no more than two α-globin genes, in particular, the at least two α-globin genes being on different chromosomes.

Accordingly, in a particular embodiment, a cell as described herein comprises in at least one of its α-globin genes comprised in the genome thereof, at least one transgene encoding a functional β-like globin protein under the control of the endogenous sequence allowing the transcription and/or enhancing the transcription, in particular the promoter, of the said at least one the α-globin gene,

the at least one the α-globin gene being selected from the group consisting of the α1-globin gene and the α2-globin gene; and

the at least one transgene encoding a functional β-like globin protein being comprised in the 5′ region, in the first intron (IVS1) and/or in the second intron (IVS2) of the said α-globin gene, in particular in the 5′ UTR and/or in the proximal promoter and/or in the second intron (IVS2) of the said α-globin gene, preferably in the 5′ UTR or in the proximal promoter or in the second intron (IVS2) of the said α-globin gene.

According to a particular embodiment, a cell according to the invention is such that the at least one α-globin gene comprised in the genome of the said HSC is the α1-globin gene and/or the α2-globin gene and the at least one transgene encoding a functional β-like globin protein is comprised in the 5′ untranslated region (5′UTR) or in an intron, in particular is comprised in the 5′ untranslated region (5′UTR) or in the second intron (IVS2), of the said at least one α-globin gene.

The present invention also relates to a blood cell, or erythroid cells, preferably purified, originating from a genetically modified stem cell as described herein.

By “originating from”, it is meant that, following the process of cellular differentiation, a genetically modified stem cell has differentiated into a blood cell or an erythroid cell.

The modified stem cell is in particular selected from the group consisting of a HSC and an ESC and is preferably a HSC. In a preferred embodiment, a blood cell as described herein is a cell from the hematopoietic system.

HSCs can differentiate into two types of progenitor cells, i.e. in myeloid progenitors or in lymphoid progenitors. While the myeloid progenitor will differentiate into Megakaryocytes, Thrombocytes, Erythrocytes, Mast cells, Myeloblasts, Basophils, Neutrophils, Eosinophils, Monocytes and Macrophages, the Lymphoid progenitor can differentiate into natural killer cells (NK), small lymphocytes, T lymphocytes, B lymphocytes and plasma cells.

Accordingly, a “blood cell” as described herein can be selected from the group consisting of lymphoid progenitors, myeloid progenitors, megakaryocytes, thrombocytes, erythrocytes, mast cells, myeloblasts, basophils, neutrophils, eosinophils, monocytes, macrophages, natural killer cells, small lymphocytes, T lymphocytes, B lymphocytes and plasma cells.

The HSC and the lymphoid progenitors and myeloid progenitors will not express the transgene, but are still useful as they can differentiate into cells that are able to do so.

In a preferred embodiment, a blood cell as described herein is selected from the group consisting of megakaryocytes, thrombocytes, erythrocytes, mast cells, myeloblasts, basophils, neutrophils, eosinophils, monocytes, macrophages, natural killer cells, small lymphocytes, T lymphocytes, B lymphocytes and plasma cells.

### Transgene

A transgene as described herein encodes at least one β-like globin protein and/or at least one β-like globin ribonucleic acid, and in particular encodes at least one functional β-like globin protein.

A β-like globin gene according to the invention refers to a gene selected from the group consisting of epsilon-globin (c), gamma-G-globin (G γ), gamma-A-globin (A γ), delta-globin (δ) and beta-globin (β) genes. In particular, a β-like globin gene according to the invention is a β-globin gene.

Accordingly, a β-like globin protein according to the invention refers to a protein selected from the group consisting of ε-globin, G-γ-globin, A-γ-globin, δ-globin and β-globin proteins. In particular, a β-like globin protein according to the invention is a β-globin protein.

A β-like globin gene according to the invention encompasses β-like globin gene of natural or artificial origin. By a β-like globin gene of artificial origin is meant a β-like globin gene obtained by artificial gene synthesis technics well known to the man skilled in the art.

The transgene encoding a functional β-like globin protein in a cell as described herein is under the control of an endogenous sequence allowing the transcription and/or enhancing the transcription of the α-globin gene comprising the said transgene.

An endogenous sequence allowing the transcription and/or enhancing the transcription of the α-globin gene may be selected from an endogenous promoter or from an endogenous enhancer of the α-globin gene.

An endogenous promoter of an α-globin gene is a sequence that initiates the transcription of the α-globin gene.

An endogenous enhancer of an α-globin gene is a short sequence of α-globin gene that can be bound by activator proteins to increase the likelihood of transcription of the α-globin gene.

In a particular embodiment, the transgene encoding a functional β-like globin protein in a cell of the invention is under the control of the endogenous promoter of the α-globin gene comprising the said transgene.

In an embodiment, a cell according to the invention can comprise only one transgene in one of its α-globin genes, the transgene encoding only one functional β-like globin protein as described herein.

In another embodiment, a cell according to the invention can comprise only one transgene in one of its α-globin genes, the transgene encoding more than one functional β-like globin protein as described herein, in particular two, three or four, functional β-like globin proteins, preferably two functional β-like globin proteins.

When there is more than one functional β-like globin protein encoded by a transgene as described herein, the functional β-like globin proteins can be identical or different one from the other.

Moreover, when there is more than one β-like globin transgene in a cell according to the invention, the said transgenes can independently be in the same or in a different α-globin gene, and, if in the same α-globin gene, in the same or in a different part of the α-globin gene as described above.

In another embodiment, a cell according to the invention can comprise more than one transgene in at least one of its α-globin genes, in particular two, three or four transgenes.

According to this embodiment, the different transgenes can independently encode the same or different functional β-like globin proteins from one transgene to another.

Also according to this embodiment, the different transgenes can independently encode one, or more than one, functional β-like globin proteins as described herein. They preferably encode only one functional β-like globin protein.

In particular, all the transgenes can encode only one functional β-like globin protein. Alternatively, at least one, in particular all the transgenes can encode more than one, in particular two, three or four, more preferably two, functional β-like globin proteins.

Moreover, the functional β-like globin proteins encoded by the different transgenes can independently be identical or different.

Still according to this embodiment, the transgenes in a cell according to the invention can independently be in the same or in a different α-globin gene, and, if in the same α-globin gene, in the same or in a different part of the α-globin gene.

A transgene as described herein can encode any type of functional β-like globin protein.

The functional β-like globin protein encoding transgene can be in a wild-type form or a codon-optimized form. Such optimized sequences can advantageously allow higher transgene expression and protein production.

In a particular embodiment, a transgene as described herein has a size inferior to 15 kb, more particularly inferior to 10 kb, preferably inferior to 8 kb.

In a particular embodiment, a transgene as described herein further comprises another gene of interest, said other gene of interest being different:
from a gene encoding a functional α-globin protein; and
from a gene encoding for a functional β-like globin gene.

### Method for the Preparation of a Hematopoietic Stem Cell (HSC) According to the Invention

A method for the preparation of a modified HSC according to the invention is ex vivo or in vitro.

The present invention indeed in particular relates to a method for the ex vivo or in vitro preparation of a HSC according to the invention, the method comprising the steps of:

(i) providing to the said stem cell a site-directed genetic engineering system by:
(a) providing to the said stem cell (1) at least one guide nucleic acid binding to a selected target site or (2) at least one guide peptide-containing endonuclease binding to a selected target site, the said target site being located in an endogenous α-globin gene comprised in the genome of the said HSC;
(b) when the at least one guide nucleic acid has been provided at step (a) (1), further providing to the said stem cell at least one endonuclease devoid of target site specificity; and
(c) further providing to the said stem cell at least one transgene that encodes a functional β-like globin protein;

and

(ii) culturing the stem cell obtained at step (i) such that the said at least one transgene encoding a functional β-like globin protein is introduced at the said selected target site in the genome of the said HSC and placed under the control of the endogenous sequence allowing the transcription and/or enhancing the transcription of the said at least one α-globin gene, in particular the endogenous promoter of the said at least one α-globin gene.

A target site as described herein can be present in a domain of an α-globin gene as defined above, the said domain being selected from the group consisting of the 5′ region, an exon domain, an intron domain and the 3 ‘UTR domain of the α-globin gene, in particular from the group consisting of the 5’ region, an exon domain and an intron domain of the said α-globin gene, more particularly from the group consisting of the 5′ UTR, of the proximal promoter and an intron domain of the said α-globin gene.

In a preferred embodiment, the said target site is located in the 5′ region and/or in an intron of the said at least one α-globin gene, preferably in the 5′ untranslated region (5′ UTR) and/or in the proximal promoter and/or in the second intron (IVS2) of the said at least one α-globin gene, in particular is comprised in the 5′ untranslated region (5′ UTR) or in the proximal promoter or in the second intron (IVS2) of the said at least one α-globin gene.

In a particular embodiment of this method, the HSC used present a β-hemoglobinopathy phenotype, i.e. present a diminished expression of β-like globin compared to a healthy corresponding cell. In particular, the HSC cells genetically modified according to the invention present a β-thalassemia or sickle-cell disease phenotype, in particular a β-thalassemia phenotype.

In a particular embodiment, step (i) (a) of a method as described herein is defined as providing to the said stem cell a guide peptide-containing endonuclease binding to a selected target site. According to the invention, the said target is located in an endogenous α-globin gene comprised in the genome of the said HSC. As such, the terms “selected target site” used herein, and unless otherwise defined, refer to a target being located in an endogenous α-globin gene in the genome of the cell.

In another embodiment, step (i) (a) of a method as described herein is defined as providing to the said stem cell at least one guide nucleic acid (or gRNA) binding to a selected target site.

gRNAs are target-specific short single-stranded RNA sequences with an 80-nucleotide constant region and a short 20-nucleotide target-specific sequence (in 5′ of the gRNA sequence) that binds to a DNA target via Watson-Crick base pairing.

In a particular embodiment, step (i) (a) of a method as described herein is defined as providing to the said stem cell two guide nucleic acids (or gRNAs) binding to two different target sites in the same globin gene domain.

As defined above, step (i) (b) of a method as described herein is only present when step (i) (a) of the said method as described herein is defined as providing to the said stem cell at least one guide nucleic acid (or gRNA) binding to a selected target site.

An endonuclease as described herein is defined as being devoid of target site specificity, i.e. the said endonuclease is not able to recognize by itself a specific target site in the genome of the HSC described herein.

In order to specifically cleave DNA at a particular target site, such endonuclease needs to be associated to a guide nucleic acid binding to the selected target site or to a guide peptide. According to the invention, and as mentioned above, the selected target site is located in an endogenous α-globin gene comprised in the genome of the said HSC When associated to a guide peptide, a guide peptide-containing endonuclease is mentioned herein.

When guided to the target site by the guide peptide or guide nucleic acid (gRNA), an endonuclease as described herein is able to introduce a single-stranded break or a double-stranded break in the said target site.

In particular, when a single guide nucleic acid molecule (gRNA) binding to a selected target site or a guide peptide-containing endonuclease binding to a selected target site is provided in step (i)(a) of the method as described herein, then the endonuclease part of the guide peptide-containing endonuclease or the endonuclease of step (i)(b) of the method as defined herein is able to introduce a double-stranded break at the target site.

In another embodiment, when two guide nucleic acid molecules (gRNAs) able to recognize two different target sites in the domain of the globin gene are provided in step (i)(a), one or two endonuclease(s) devoid of target site specificity can be provided in step (i)(b) of the method as described herein, the one or two endonuclease(s) being able to introduce a single-stranded break at the two different target sites.

Steps (i)(a), (i)(b) and (i)(c) of the method as described herein can be independently realized simultaneously or separately from one another. In a preferred embodiment, the at least one guide nucleic acid binding to a selected target site or the guide peptide-containing endonuclease binding to a selected target site of step (i)(a), the at least one endonuclease devoid of target site specificity of step (i)(b) and the transgene of step (i)(c) are provided simultaneously to the said stem cell.

Methods to introduce in vitro, ex vivo or in vivo proteins and nucleic acid molecules into cells are well known in the art. The traditional methods to introduce a nucleic acid, usually present in a vector, or a protein in a cell include microinjection, electroporation and sonoporation. Other techniques based on physical, mechanical and biochemical approaches such as magnetofection, optoinjection, optoporation, optical transfection and laserfection can also be mentioned (see Stewart M P et al., Nature, 2016).

In a particular embodiment, the endonuclease devoid of target site specificity of step (i)(b) is a RNA-guided endonuclease. In particular, this RNA-guided endonuclease can be directed by the gRNA to introduce a single- or a double-stranded break within at the target site.

A RNA-guided endonuclease as described herein can in particular be a Clustered regularly interspaced short palindromic repeats (CRISPR) associated protein, (Cas), in particular the CRISPR associated protein 9 (Cas9) or the CRISPR-associated endonuclease in Lachnospiraceae, Acidaminococcus, Prevotella and Francisella 1 (Cpf1).

In a particular embodiment, the at least one endonuclease devoid of target site specificity is a Clustered regularly interspaced short palindromic repeats (CRISPR) associated nuclease, in particular the CRISPR associated protein 9 (Cas9).

CRISPR-Cas systems for genome editing are particular systems using simple base pairing rules between an engineered RNA and the target DNA site instead of other systems using protein-DNA interactions for targeting.

CRISPR-Cas RNA-guided nucleases are derived from an adaptive immune system that evolved in bacteria to defend against invading plasmids and viruses.

According to a first embodiment, it consists in a mechanism by which short sequences of invading nucleic acids are incorporated into CRISPR loci. They are then transcribed and processed into CRISPR RNAs (crRNAs) which, together with a trans-activating crRNAs (tracrRNAs), complex with CRISPR-associated (Cas) proteins to dictate specificity of DNA cleavage by Cas nucleases through Watson-Crick base pairing between nucleic acids. The crRNA harbors a variable sequence known as the “protospacer” sequence. The protospacer-encoded portion of the crRNA directs Cas9 to cleave complementary target DNA sequences if they are adjacent to short sequences known as “protospacer adjacent motifs” (PAMs). Protospacer sequences incorporated into the CRISPR are not cleaved because they are not present next to a PAM sequence (see Mali et al. (Nat. Methods, 2013 October; 10(10):957-63); and Wright et al. (2016 Jan. 14; 164(1-2):29-44 Cell).

According to this first embodiment, a guide RNA (gRNA) as described herein either corresponds to a single RNA (and is then called sgRNA) or corresponds to the fusion of the crRNA and tracrRNA. The term guide RNA or gRNA used in the present text designates these two forms, except when a particular form is specifically indicated.

In a gRNA according to this embodiment and corresponding to the fusion of the crRNA and tracrRNA, nucleotides 1-32 are the naturally-occurring crRNA while nucleotides 37-100 are the naturally-occurring tracrRNA, nucleotides 33-36 corresponding to a GAAA linker between the two pieces of gRNA (see Jinek et al. (2012) Science 337:816-821 and Cong et al. (2013) Science; 339(6121): 819-823).

Such gRNA is advantageously used in a CRISPR-Cas9 system.

gRNA are artificial and do not exist in nature.

The sequence of a gRNA, as indicated above, is a RNA sequence complementary to its targeted DNA sequence.

Preferred gRNA as described herein can be selected among gRNA comprising a nucleic sequence complementary to a nucleic sequence selected from the group consisting of SEQ ID NO: 1 to SEQ ID NO: 32. Particularly preferred gRNA as described herein can be selected among gRNA comprising a nucleic sequence complementary to a nucleic sequence selected from the group consisting of SEQ ID NO: 1 to SEQ ID NO: 14, SEQ ID NO: 31 and SEQ ID NO: 32, more preferably comprising a nucleic sequence complementary to a nucleic sequence selected from the group consisting of SEQ ID NO: 3, SEQ ID NO: 4 and SEQ ID NO: 7 to SEQ ID NO: 16.

More particularly, gRNA as described herein are selected among gRNA comprising a nucleic sequence complementary to a nucleic sequence selected from the group consisting of SEQ ID NO: 3, SEQ ID NO: 4 and SEQ ID NO: 7 to SEQ ID NO: 10.

In particular, a gRNA used in a method of the invention is the one having a sequence complementary to the nucleic sequence SEQ ID NO: 7 or 8, in particular SEQ ID NO: 7.

Other gRNAs, that can be used with different Cas or Cas9 nucleases having different PAMs, can be designed and used in a method according to the invention.

The said nucleic acid sequence of 20 nucleotides corresponds to the first 20 nucleotides at the 5′ end of the full gRNA sequence and, in the present first embodiment, directly precedes the PAM sequence, preferably the PAM sequence NGG, i.e. the PAM signal is nucleotides 21-23 of the full sequence of the sequences Table.

In a PAM sequence, the N can be an adenine (A), a cytosine (C), a thymine (T) or a guanine (G).

As illustrated in the examples, among many designed gRNA targeting either the 5′ region (5′UTR or proximal promoter) or the introns (IVS1 or IVS2) of HBA1 and/or

HBA2, the inventors selected specific gRNA for minimizing the possibility of generating an allele KO if the Cas9-induced Double Strain Break (DSB) doesn't result in a positive dDNA integration event.

According to another embodiment, the mechanism is similar to the one of the first embodiments mentioned above, except that no trans-activating crRNAs (tracrRNAs) are used. Indeed, in this embodiment, CRISPR RNAs (crRNAs) complex with CRISPR-associated (Cas) proteins to dictate specificity of DNA cleavage by Cas nucleases through Watson-Crick base pairing between nucleic acids. According to this embodiment, the Cas protein is advantageously Cpf1. Indeed, an important difference between Cpf1 and Cas9 for example is that Cpf1 is a single-RNA-guided nuclease that does not require a tracrRNA.

The Cpf1 enzyme has been isolated from the bacteria Francisella novicida. The Cpf1 protein contains a predicted RuvC-like endonuclease domain that is distantly related to the respective nuclease domain of Cas9. However, Cpf1 differs from Cas9 in that it lacks HNH, a second endonuclease domain that is present within the RuvC-like domain of Cas9. Cpf1 recognizes a T-rich PAM, TTTN on the 5′ side of the guide, which makes its distinct from Cas9 which uses a NGG PAM on the 3′ side of the guide.

In the PAM sequence TTTN, the N can be an adenine (A), a cytosine (C), a thymine (T) or a guanine (G).

According to the present embodiment, a guide RNA (gRNA) as described herein corresponds to a sole crRNA and does not need to be fused with tracrRNA. Such gRNA is advantageously used in a CRISPR-Cpf1 system.

According to a particular embodiment, guide peptide-containing endonuclease binding to a selected target site of step (i)(a) of a method defined herein is a transcription activator-like effector nuclease (TALEN) or a zinc-finger nuclease.

The TALENs technology comprises a non-specific DNA-cleaving domain (nuclease) fused to a specific DNA-binding domain. The specific DNA-binding domain is composed of highly conserved repeats derived from transcription activator-like effectors (TALEs) which are proteins secreted by Xanthomonas bacteria to alter transcription of genes in host plant cells. The DNA-cleaving domain or cleavage half-domain can be obtained, for example, from various restriction endonucleases and/or homing endonucleases (for example Fok I Type IIS restriction endonuclease) of Fok I. (see Wright et al. (Biochem. J. 2014 Aug. 15; 462(1):15-24)).

The zinc-finger nuclease (ZFN) technology consists in the use of artificial restriction enzymes generated by fusion of a zinc finger DNA-binding domain to a DNA-cleavage domain (nuclease). The zinc finger domain specifically targets desired DNA sequences, which allows the associated nuclease to target a unique sequence within complex genomes.

The zinc finger DNA-binding domain comprises a chain of two-finger modules, each recognizing a unique hexamer (6 bp) sequence of DNA. The two-finger modules are stitched together to form a Zinc finger protein. As in the TALENs technology, the DNA-cleavage domain comprises the nuclease domain of Fok I (Carroll D, Genetics, 2011 August; 188(4): 773-782; Urnov F. D. Nat Rev Genet. (9):636-46, (2010)).

Concerning the transgene that encodes a functional β-like globin protein of step (i)(c) of the method as described herein, the said transgene is preferably not under the control of a promoter and/or the provided transgene is under the control of a promoter that will not be inserted in the genome of the stem cell.

Indeed, as previously indicated, a genetically modified HSC as described herein and that can be obtained from a method as defined previously is such that the at least one transgene encoding a functional β-like globin protein comprised in an α-globin gene is under the control of the endogenous sequence allowing the transcription and/or enhancing the transcription, in particular the promoter, of the said α-globin gene in which it is inserted.

The use of the endogenous sequence allowing the transcription and/or enhancing the transcription, in particular the promoter, of an α-globin gene of a HSC according to the invention advantageously allows high and tissue specific transcription of the transgene(s) in the cell as mentioned above.

In step (ii) of a method as described herein, the stem cell obtained at step (i) is cultured such that the said transgene is introduced at the said selected target site in the genome of the said HSC.

In a particular embodiment, the method for the ex vivo or in vitro preparation of a HSC according to the present invention comprises the steps of:

(i) providing to the said stem cell a site-directed genetic engineering system by:
(a) providing to the said stem cell at least one guide nucleic acid binding to a selected target site, the said target site being located in an α-globin gene comprised in the genome of the said HSC;
(b) further providing to the said stem cell at least one endonuclease devoid of target site specificity; and
(c) further providing to the said stem cell at least one transgene that encodes a functional β-like globin protein;

and

(ii) culturing the stem cell obtained at the end of step (i) such that the said at least one transgene is introduced at the said selected target site in the genome of the said HSC.

As mentioned previously, the at least one α-globin gene is preferably selected from the group consisting of the α1-globin gene and the α2-globin gene.

In a particular embodiment, the present invention relates to method for the ex vivo or in vitro preparation of a HSC according to the invention, comprising the steps of:

(i) providing to the said stem cell a site-directed genetic engineering system by:
(a) providing to the said stem cell at least one guide nucleic acid binding to a selected target site, the said target site being located in the 5′ region and/or in the second intron (IVS2) of an endogenous α-globin gene comprised in the genome of the said HSC;
(b) further providing to the said stem cell at least one Clustered regularly interspaced short palindromic repeats (CRISPR) associated nuclease, in particular the CRISPR associated protein 9 (Cas9); and
(c) further providing to the said stem cell at least one transgene that encodes a functional β-like globin protein;

and

(ii) culturing the stem cell obtained at the end of step (i) such that the said transgene is introduced at the said selected target site in the genome of the said HSC.

In a particular embodiment, the present invention relates to method for the ex vivo or in vitro preparation of an HSC according to the invention comprising the steps of:

(i) providing to the said stem cell a site-directed genetic engineering system by:
(a) providing to the said stem cell at least one guide nucleic acid binding to a selected target site, the said target site being located in the 5′ region and/or in the second intron (IVS2) of an endogenous α-globin gene comprised in the genome of the said HSC;
(b) further providing to the said stem cell at least one Clustered regularly interspaced short palindromic repeats (CRISPR) associated nuclease, in particular the CRISPR associated protein 9 (Cas9); and
(c) further providing to the said stem cell at least one transgene that encodes a functional β-like globin protein;

and

(ii) culturing the stem cell obtained at the end of step (i) such that the said transgene is introduced at the said selected target site in the genome of the said HSC,

the said at least one guide nucleic acid binding to a selected target site selected from the group consisting of gRNAs consisting in a nucleic sequence complementary to a nucleic sequence selected from the group consisting of SEQ ID NO: 1 to SEQ ID NO: 32, preferably selected from the group consisting of SEQ ID NO: 1 to SEQ ID NO: 14, SEQ ID NO: 31 and SEQ ID NO: 32, more particularly from the group consisting of SEQ ID NO: 3, SEQ ID NO: 4 and SEQ ID NO: 7 to SEQ ID NO: 16.

### Pharmaceutical Composition

The present invention also relates to a pharmaceutical composition comprising at least one genetically modified HSC as described herein and/or at least one blood cell (or erythroid cell) as described herein in a pharmaceutically acceptable medium.

A pharmaceutically acceptable medium as described herein is in particular suitable for administration to a mammalian individual.

A “pharmaceutically acceptable medium” comprises any of standard pharmaceutically accepted mediums known to those of ordinary skill in the art in formulating pharmaceutical compositions, for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxym ethyl cellulose, hydroxylpropyl methyl cellulose, magnesium stearate, calcium phosphate, gelatine or polysorbate 80 or the like.

A pharmaceutical composition as described herein will often further comprise one or more buffers (e.g., neutral buffered saline or phosphate buffered saline); carbohydrates (e.g., glucose, mannose, sucrose or dextrans); mannitol; proteins; polypeptides or amino acids such as glycine; antioxidants (e.g., ascorbic acid, sodium metabisulfite, butylated hydroxytoluene, butylated hydroxyanisole, etc.); bacteriostats; chelating agents such as EDTA or glutathione; solutes that render the formulation isotonic, hypotonic or weakly hypertonic with the blood of a recipient; suspending agents; thickening agents and/or preservatives.

Of course, the type of carrier will typically vary depending on the mode of administration.

In an embodiment, the cells as described herein can be used in a composition in combination with therapeutic compounds that are effective in treating the conditions associated with hemoglobinopathies, in particular with β-thalassemias, in the individual in need thereof. For example, a cell as described herein can be administered with antibacterial, antifungal, or antiviral compounds for preventing opportunistic infections or infections already in progress in the individual. Illustratively, platelets can be administered together with cells as described herein in a composition as described herein as a temporary measure to restore platelet count to safe levels.

In an embodiment, the cells as described herein can be used in a composition as described herein in combination with other HSC or blood cells as defined above, but not modified as described herein.

In an embodiment, the cells as described herein can be used in a composition as described herein in combination with other agents and compounds that enhance the therapeutic effect of the administered cells.

In another embodiment, the cells as described herein can be administered in a composition as described herein with therapeutic compounds that enhance the differentiation of the HSC or progenitor cells as described herein. These therapeutic compounds have the effect of inducing differentiation and mobilization of HSC and/or of progenitor cells that are endogenous, and/or the ones that are administered to the individual as part of the therapy.

Genetically Modified Cells for their Use as a Medicament

Another object of the present invention is a HSC according to the invention, or a blood cell according to the invention, or a pharmaceutical composition according to the invention, for its use as a medicament.

Cells as described herein are administered into a subject by any suitable route, such as intravenous, intracardiac, intrathecal, intramuscular, intra-articular or intra-bone marrow injection, and in a sufficient amount to provide a therapeutic benefit.

The amount of cells needed for achieving a therapeutic effect will be determined empirically in accordance with conventional procedures for the particular purpose.

Generally, for administering the cells for therapeutic purposes, the cells are given at a pharmacologically effective dose.

By “pharmacologically effective amount” or “pharmacologically effective dose” is meant an amount sufficient to produce the desired physiological effect or amount capable of achieving the desired result, particularly for treating the disorder or disease condition, including reducing or eliminating one or more symptoms or manifestations of the disorder or disease.

Illustratively, administration of cells to a patient suffering from a β-thalassemia provides a therapeutic benefit when the amount of β-like globin, and therefore the amount of hemoglobin, in the patient is increased, when compared to the amount of β-like globin, and therefore the amount of hemoglobin, in the patient before administration.

Cells are administered by methods well known in the art. In one embodiment, the administration is by intravenous infusion. In another method, the administration is by intra-bone marrow injection.

The number of cells transfused will take into consideration factors such as sex, age, weight, the types of disease or disorder, stage of the disorder, the percentage of the desired cells in the cell population (e.g., purity of cell population), and the cell number needed to produce a therapeutic benefit.

Generally, the numbers of cells infused may be from 1.10⁴ to 5.10⁶ cells/kg, in particular from 1.10⁵ to 10.10⁶ cells/kg, preferably from 5.10⁵ cells to about 5.10⁶ cells/kg of body weight.

A pharmaceutical composition as described herein, as previously mentioned, can be used for administration of the cells as described herein into the individual in need thereof.

The administration of cells can be through a single administration or successive administrations. When successive administrations are involved, different cells numbers and/or different cells populations may be used for each administration.

Illustratively, a first administration can be of a cell or a cell population as described herein that provides an immediate therapeutic benefit as well as more prolonged effect, while the second administration includes cells as described herein that provide prolonged effect to extend the therapeutic effect of the first administration.

A stem cell, a blood cell or a pharmaceutical composition as described herein can be used in the treatment of hemoglobinopathies, in particular of β-hemoglobinopathies, in particular for use in the treatment of β-thalassemia in an individual in need thereof.

In a particular embodiment, a stem cell, a blood cell or a pharmaceutical composition as described herein can be used in the treatment of a severe for of β-thalassemia, also known as β-thalassemia major, in an individual in need thereof.

Accordingly, a further object of the invention is a genetically modified HSC as described herein, or a blood cell as described herein, or a pharmaceutical composition as described herein, for its use in the treatment of hemoglobinopathies, in particular of β-hemoglobinopathies, in particular for use in the treatment of β-thalassemia, in particular of β-thalassemia major, in an individual in need thereof.

It can also be mentioned a method for the treatment of hemoglobinopathies, in particular of β-hemoglobinopathies, in particular for use in the treatment of β-thalassemia, in particular of β-thalassemia major, in an individual in need thereof comprising the administration of a HSC as described herein, a blood cell of the invention, and/or a pharmaceutical composition as described herein to said individual in need thereof.

The present invention also relates to the use of a HSC as described herein, a blood cell of the invention, and/or a pharmaceutical composition as described herein for the manufacture of a medicament for treating hemoglobinopathies, in particular β-hemoglobinopathies, in particular for treating β-thalassemia, in particular of β-thalassemia major, in an individual in need thereof

### Method for Restoring the Physiological Level of Production of Hemoglobin in a Cell

Another object of the present invention is an in vitro or ex vivo method for restoring the physiological level of production of hemoglobin in a cell, in particular in a blood cell, more particularly a red blood cell, whose β-like globin expression, and in particular β-globin expression, is defective, comprising at least the steps of:

(i) providing to the cell a site-directed genetic engineering system by:
(a) providing to the said cell (1) at least one guide nucleic acid binding to a selected target site or (2) at least one guide peptide-containing endonuclease binding to a selected target site, the said target site being located in an endogenous α-globin gene comprised in the genome of the said cell;
(b) when the at least one guide nucleic acid has been provided at step (a) (1), further providing to the said cell at least one endonuclease devoid of target site specificity; and
(c) further providing to the said cell at least one transgene that encodes a functional β-like globin protein;

(ii) culturing the cell obtained at step (i) such that the said at least one transgene encoding a functional β-like globin protein is introduced at the said selected target site in the genome of the said cell and placed under the control of the endogenous sequence allowing the transcription and/or enhancing the transcription of the said at least one α-globin gene; and

(iii) culturing the cell obtained at step (ii) such that the at least one transgene encoding a functional β-like globin protein that has been introduced in the genome of the cell is transcribed by the endogenous transcription system of the cell.

In a particular embodiment, steps (i) and (ii) of the in vitro or ex vivo method for restoring the physiological level of production of hemoglobin in a cell whose β-like globin expression is defective according to the invention, may be defined in the same manner as steps (i) and (ii) from the method for the preparation of a HSC as described above.

In particular, “restoring the physiological level of production of hemoglobin” means that the level of production of hemoglobin in a cell, in particular a red blood cell, originating from a HSC having undergone the method herein is the same, or at least similar, to the level of production of hemoglobin in a cell, in particular a red blood cell, whose β-globin expression is not defective.

In particular, the restored level of hemoglobin in the cell which has undergone the method herein is the same, or at least very similar, to the level of hemoglobin in a cell, in particular in a blood cell, more particularly a red blood cell, from an individual who does not suffer from β-hemoglobinopathies, in particular who does not suffer from β-thalassemia.

In particular, the method for restoring the physiological level of production of hemoglobin herein also allows restoring the physiological level of production of β-like globin, and in particular of β-globin, in a cell, in particular in a blood cell, more particularly a red blood cell, whose β-like globin expression, and in particular β-globin expression, is defective.

In a particular embodiment, “restoring the physiological level of production of β-like globin” means that the level of production of β-like globin in the cell after undergoing the method herein is the same, or at least similar, to the level of β-like globin in a cell, in particular in a blood cell, more particularly a red blood cell, whose β-like globin expression is not defective. As such, the level of β-like globin in the cell after undergoing the method herein is significantly higher than the level of β-like globin in the cell before undergoing the method herein.

In particular, the restored level of β-like globin in the cell which has undergone the method herein is the same, or at least similar, to the level of β-like globin in a cell, in particular in a blood cell, more particularly a red blood cell, from an individual who does not suffer from β-hemoglobinopathies, in particular who does not suffer from β-thalassemia.

A cell, in particular in a blood cell, more particularly a red blood cell, whose β-like globin expression is defective according to the invention means a cell whose β-like globin expression level is such that the α/β-like globin chain balance, and in particular the α/β-globin chain balance, in the cell leads to an accumulation and precipitation of free α-globin chains.

As such, the β-like globin expression in the cell, in particular in a blood cell, more particularly a red blood cell, originating from a genetically modified HSC according to the invention is no longer considered as defective, once the α/β-like globin chain balance in the cell no longer leads to an accumulation and precipitation of free α-globin chains.

An example of a cell, in particular in a blood cell, more particularly a red blood cell, whose β-like globin expression is defective, is a cell from an individual suffering from hemoglobinopathies, in particular from an individual suffering from β-thalassemia.

### Kit for Restoring In Vitro or Ex Vivo the Physiological Level of Production of Hemoglobin in a Cell

The present invention also relates to a kit providing elements necessary to perform the methods of the invention as defined above.

In particular, the present invention relates to a kit for genetically editing a HSC in order for it to be able to differentiate into a cell, in particular a blood cell, and more particularly a red blood cell, having a restored physiological level of production of hemoglobin, in particular a level of production of hemoglobin which is similar to a cell, in particular a blood cell, more particularly a red blood cell, whose β-globin expression is not defective.

Accordingly, another object of the invention relates to a kit for restoring in vitro or ex vivo the physiological level of production of hemoglobin in a cell, in particular in a blood cell, more particularly a red blood cell, comprising:

(i) at least one guide nucleic acid binding to a selected target site or at least one guide peptide-containing endonuclease binding to a selected target site, the said target site being located in an endogenous α-globin gene comprised in the genome of a HSC;

(ii) when the at least one guide nucleic acid is provided as part (i), further at least one endonuclease devoid of target site specificity;

(iii) at least one transgene encoding a functional β-like globin protein, in particular a functional β-globin protein.

Every single one of the elements constituting a kit according to the invention can be as previously detailed in the present text.

In a particular embodiment, a kit according to the invention additionally comprises the HSC which must undergo the genetic editing mentioned above.

The present invention is further illustrated by, without in any way being limited to, the examples herein.

### EXAMPLES

### Example 1: Design and Validation of gRNAs in K562 Erythroleukemia Cell Line

The inventors designed several gRNA targeting the 5′ region (5′ UTR or proximal promoter), the 3′ untranslated region (3′UTR) or one of the introns (IVS1 or IVS2) of HBA1/2 genes.

gRNA candidates encoding plasmids were nucleofected in a stable K562 cell clone constitutively expressing SpCas9 (K562-Cas9).

The gRNA candidates used in this example are those represented in the Table indicated later in the present text.

More particularly, K562 (ATCC® CCL-243) were maintained in RPMI 1640 medium (Gibco) containing 2 mM glutamine and supplemented with 10% fetal bovine serum (FBS, BioWhittaker, Lonza), HEPES (10 mM, LifeTechnologies), sodium pyruvate (1 mM, LifeTechnologies) and penicillin and streptomycin (100 U/ml each, LifeTechnologies). A stable clone of K562-Cas9 was made by infection with a lentiviral vector (Addgene #52962) expressing spCas9 and a blasticidin resistance cassette, selected and subcloned.

2.5×10⁵ of K562-Cas9 cells were transfected with 200 ng of gRNA-containing vector (Addgene #53188) in a 204, volume using Nucleofector Amaxa 4D (Lonza) with SF Cell Line 4D-Nucleofectof Kit.

48 hours after nucleofection, cells were pelleted, and DNA was extracted using MagNA Pure 96 DNA and Viral NA Small Volume Kit (Roche). 50 ng of genomic DNA were used to amplify the region that spans the cutting site of each gRNA using KAPA2G Fast ReadyMix (Kapa Biosystem). After Sanger sequencing, the percentage of Inversions and Deletions (InDel) was calculated by the software TIDE (Tracking of InDels by Decomposition—Brinkman et al. Nucleic Acids Res. 2014. 42(22):e168)) using the website www.tide.calculator.nk.

The InDel is the insertion or deletion of bases in the genome caused by non-homologous end joining (NHEJ) DNA repair of the DNA ends generated by the nuclease activity. It is well defined in Brinkman et al. (Brinkman et al. Nucleic Acids Res. 2014. 42(22):e168)). Accordingly, the closer to 100% an InDel result, the more efficient the gRNA tested.

The results obtained are presented in FIG. 1.

The best performing gRNA for target 5′ region, in particular the 5′UTR, 3′UTR, IVS1 and IVS2 of HBA (respectively the gRNA having the sequence complementary to the nucleic sequence SEQ ID NO: 8 for 5′ region, the gRNA having the sequence complementary to the nucleic sequence SEQ ID NO: 28 for IVS1 and the gRNA having the sequence complementary to the nucleic sequence SEQ ID NO: 32 for IVS2) were further tested for integration and expression of transgene containing donor DNA (dDNA). As dDNA, integrase-defective lentiviral vectors (IDLV) encoding for a promoterless GFP were generated, expected to be under the control of the endogenous α globin promoter upon successful targeting, and a puromycin expression cassette, to enrich for dDNA containing cells.

To test dDNA integration, K562-Cas9 cells were first transduced with Integrase-Deficient Lentiviral Vectors (IDLV) and 24 h later transfected with the selected gRNA-encoding plasmids. After puromycin selection, a high percentage of GFP positive cells were observed for all gRNA and dDNA tested (data not shown) while no significant GFP expression was detected upon random integration of the cassette (not shown).

The inventors then induced erythroid differentiation of K562 to upregulate HBA and HBB transcription and observed a concomitant increase in GFP expression, confirming that the reporter was indeed under the transcriptional regulation of endogenous globin promoters (data not shown). Finally, correct integration for all gRNA/dDNA combinations was validated by PCR and Sanger sequencing on single GFP+ cell clones (data not shown).

In summary, the inventors identified several efficient gRNA and designed dDNA cassettes to achieve precise and functional transgene targeted integration under the control of the endogenous erythroid α-globin promoters.

### Example 2: Generation of HBB-KO Cells as Model for Evaluating the Claimed Method and Preparation of α-Globin Deleted Clones

HBB-KO clones of a human erythroid progenitor cell line (HUDEP2) recapitulating a β0-thal phenotype (HUDEP2β0-thal) are generated to evaluate the therapeutic effect of the strategy of the present invention. HUDEP2β0-thal were generated by nucleofection of a Cas9:gRNA complex targeting exon 1 of HBB gene. Edited cells were subcloned and a single cell clone with a biallelic +1 insertion in exon 1 of HBB that generated a premature stop codon was expanded. After differentiation, absence β-globin chains or adult hemoglobin tetramers (HbA) was confirmed by HPLC.

HUDEP2 and HUDEP2 β0 cells are nucleofected with Cas9:gRNA ribonucleoprotein (RNP) targeting HBA genes. Based on the results obtained in example 1, gRNA having the sequence complementary to the nucleic sequence SEQ ID NO: 8, targeting the 5′region of HBA, is selected considering its high cleavage efficiency (˜83% InDels) that results in an average of one deletion per cell in HUDEP2 cells.

HUDEP2 and HUDEP2 β0 are cultured in maintenance media containing StemSpan Serum-Free Expansion Medium (SFEM, Stemcell Technologies), penicillin and streptomycin (100 U/ml each, LifeTechnologies), 50 ng/mL recombinant human stem cell factor (SCF), 3 IU/mL Epoetin alfa (Epogen, Amgen), 0.4 μg/mL dexamethasone, 1 μg/mL doxycycline.

Between 2×10⁵ and 2.5×10⁵ cells per condition are nucleofected with Cas9:gRNA (RNP, modified single guide RNA purchased from Synthego) complex using P3 Primary Cell 4D-Nucleofector kit (Lonza). Five days after nucleofection, the pellets are harvested and DNA is extracted using MagNA Pure 96 DNA and Viral NA Small Volume Kit (Roche). 50 ng of genomic DNA are used to amplify the region that spans the cutting site of each gRNA using KAPA2G Fast ReadyMix (Kapa Biosystem).

After Sanger sequencing, the percentage of Inversions and deletions (InDel) is calculated by the software TIDE (Tracking of InDels by Decomposition) as mentioned in example 1.

The results obtained are represented in FIG. 2.

As illustrated in this Figure, clones with α-globin deletion genotypes (Hetero (−α/αα) or Homo (−α/−α) genotypes) are efficiently generated in the two types of cells population HUDEP2 and HUDEP2 (30.

### Example 3: Observation of the Globin Balance in the Cells Generated in Example 2

The cells obtained in example 2 are differentiated for 9 days using a 3-step protocol.

The cells are maintained for 4 days in Iscove's modified Dulbecco's medium (IMDM) supplemented 1% L-glutamine, penicillin and streptomycin (100 U/ml each, LifeTechnologies), 330 μg/mL human holo-transferrin, 10 μg/mL recombinant human insulin solution, 2 IU/mL heparin, 5% inactivated human AB plasma, 3 IU/mL Epoetin alfa (Epogen, Amgen), 100 ng/mL SCF and 1 μg/mL doxycycline.

Cells are then cultured in the absence of SCF for 3 days and without doxycycline and SCF for the remaining 2 days.

Total RNA is extracted using RNeasy mini/micro Kit (QIAGEN, Germany) and treated with DNase following the manufacturer's instructions. For globin mRNA quantification, total RNA is reverse-transcribed using Transcriptor First Strand cDNA Synthesis Kit (Roche) and qPCR is performed using Syber Green/Rox (Life Scientific). Globin expression is normalized using GAPDH transcript as a reference (SEQ ID NO. 33: CTTCATTGACCTCAACTACATGGTTT, SEQ ID NO: 34: TGGGATTTCCATTGATGACAAG).

Different globin transcripts are measured: HBG1 and HBG2 (SEQ ID NO. 35: CCTGTCCTCTGCCTCTGCC; SEQ ID NO. 36: GGATTGCCAAAACGGTCAC), HBB (SEQ ID NO. 37: GCAAGGTGAACGTGGATGAAGT; SEQ ID NO. 38: TAACAGCATCAGGAGTGGACAGA), HBA (SEQ ID NO.: 39 CGGTCAACTTCAAGCTCCTAA, SEQ ID NO. 40: ACAGAAGCCAGGAACTTGTC) and HBD (SEQ ID NO. 41: CAAGGGCACTTTTTCTCAG, SEQ ID NO. 42: AATTCCTTGCCAAAGTTGC).

The results obtained are represented in FIG. 3.

The results obtained with Wild type HUDEP2 cells are also represented in this Figure (HUDEP2) as control.

Another control represented as “HUDEP2 β0 DEL KO” consists of HUDEP2 cells into which a complete knock-out of α-globin expression has been performed.

In these cells, a significant reduction of α-globin precipitates (by HPLC) upon deletion of 1 or 2 HBA genes (data not shown) and concomitant amelioration of the α/β-globin balance is observed, which correlated with HBA deletion efficiency (the α/β-like mRNA ratio goes from 6.4+/−2.4 to 2.1+/−0.6 for −α/αα cells and to 1.0+/−0.5 for −α/−α cells).

Accordingly, the globin balance has been significantly improved in these cells.

### Example 4: Observation of the Integration of a β-Globin Transgene in Thalassemic HUDEP2 β0 Cells

However, as expected for HUDEP2 β0 cells, they show lower hemoglobin levels compared to wild type cells, which is not desirable.

For this reason, the HBA deletion is combined with the knock-in of βAS3-globin transgene under the control of the endogenous HBA promoter.

βAS3-globin transgene is a recombinant human anti-sickling β-globin polypeptide described for the first time in Levasseur et al. (J Biol Chem. 2004 Jun. 25; 279(26):27518-24).

HUDEP2 β0 cells are edited with Cas9 RNP as previously described and infected with AAV6 (MOI 15000) vector illustrated in FIG. 4A containing a βAS3-globin gene and a constitutive GFP reporter between homology arms for the gRNA target site (250 bp each), for 4 hours. gRNA 15.1 was used to insert AAV in the 5′UTR of the α-globin gene.

After washing the virus, edited cells are kept in maintenance medium for 2 weeks. GFP expression is then monitored by flow cytometry as cells that stably integrated the AAV6 trap express GFP reporter 14 days after nucleofection.

The results obtained are represented in FIG. 4B show the fraction of edited HSC that expresses GFP.

The percentage of β-like globins expressed by different cells upon differentiation is also measured by HPLC.

After chromatography, peak areas corresponding to different globins are measured. Beta globin expression is represented as percentage of the total beta-like globins (beta+gamma+delta)

The results obtained are represented in FIG. 5.

Strikingly, it is observed the expression of a high level of HBB proteins in HUDEP2β0-thal cells with one integration per cell (βAS3=81.5+/−13.8% of β-like globins in edited HUDEP2β0-thal cells, versus (3=85.6+/−4.1% of β-globins in wild type HUDEP2).

It is thus demonstrated that on-target integration of a βAS3 globin transgene can satisfactorily be achieved in thalassemic HUDEP2 (30.

### Example 5: HPLC Analysis of Hemoglobin Subunits and Hemoglobin in the Genetically Engineered Cells

HUDEP2 β0 cells are edited with Cas9 RNP and infected with AAV6 as described in example 4.

After washing the virus, edited cells are kept in maintenance medium for 2 weeks.

GFP positive cells are sorted using MoFlocell sorter (Beckman Coulter) and differentiated as previously described. High performance liquid chromatography (HPLC) analysis is performed on cell pellets using a NexeraX2 SIL-30AC chromatograph (Shimadzu, Kyoto, Japan) and analyzed with LC Solution software.

Differentiated HUDEP2 β0 are lysed in water and globin chains are separated using a 250×4.6 mm, 3.6 μm Aeris Widepore column (Phenomenex). Samples are eluted with a gradient mixture of solution A (water/acetonitrile/trifluoroacetic acid, 95:5:0.1) and solution B (water/acetonitrile/trifluoroacetic acid, 5:95:0.1), monitoring absorbance at 220 nm.

The results obtained are represented in FIGS. 6a and 6b.

FIG. 6a further represents the hemoglobin subunits identified in HUDEP2 β0 cells. It can be observed that there is no production of tetramers (HBA) in these cells while toxic α-precipitates are present.

FIG. 6b represents the hemoglobin subunits identified in HUDEP2 β0 cells into which the βAS3 globin transgene has been integrated.

A high amount of HBA produced (vertical arrow) are observed, while no more α-precipitates can be observed.

Accordingly, it is demonstrated the restoration of HbA production in thalassemic HUDEP2 cells.

### Example 6: In Vivo Experiments

Protocol

NOD.Cg-Prkdc^{scid}Il2rg^{tm1Wjl}/SzJ (NSG) mice are purchased from The Jackson Laboratory (strain 005557) and maintained in specific-pathogen-free (SPF) conditions. This study is approved by ethical committee CEEA-51 and conducted according to French and European legislation on animal experimentation (APAFiS#16499-2018071809263257_v4).

Edited CD34+ cells are prepared as follows.

Human umbilical cord blood (UCB) samples are provided by Centre Hospitalier Sud-Francilien (CHSF, Evry, France) and processed according to France bioethics laws (declaration DC-2012-1655 to the French Ministry of Higher Education and Research).

CD34+ cells are purified by immunomagnetic selection with AUTOMACS PRO (Miltenyi Biotec, Paris, France) after immunostaining with CD34 MicroBead Kit (Miltenyi Biotec, Paris, France) using manufacturer's instructions. Mobilized peripheral blood (MPB) and UCB CD34+ are also purchased from Cliniscience (Nanterre, France).

MPB- or UCB-derived HSPCs are thawed and cultured in pre-stimulation media for 48 h (StemSpan, Stemregenin-1 0.75 uM, StemCell technologies, Vancouver, BC, Canada; rhSCF 300 ng/ml, rhFlt3-L 300 ng/ml, rhTPO 100 ng/ml and rhlL-3 20 ng/mL, CellGenix, Freiburg, Germany).

sgRNA (HBA15.1) are diluted following manufacturer's instructions and ribonucleoprotein complexes (RNP) are formed with 30 pmol of spCas9 (ratio 1:1.5) (spCas9 from Menoret et al., Sci Rep. 2015; 5: 14410). 2.5×10⁵ HSPCs per condition are transfected with RNP using P3 Primary Cell 4D-Nucleofector kit (CA137 program) (Lattanzi A et al. Mol Ther. 2019; 27(1):137-150). In knock-in experiments, 15 minutes after transfection, HSPCs are transduced with AAV6 detailed here-after for 6 h (MOI 10,000-30,000), washed and left in pre-stimulation media for additional 24-48 hours.

Two different cassettes are designed for the expression of β-globin gene (HBB; Gene ID: 3043):

i) the first cassette contains a β-globin cDNA, with 3 anti-sickling point mutations (β^{AS3}), followed by a woodchuck posttranscriptional regulatory element (WPRE) and a SV40polyA;

ii) the second cassette contains the whole β^{AS3} gene with introns and its original 3′ UTR and pA site, with 3 anti-sickling point mutations (Example 4).

Each cassette, followed by a GFP reporter gene under the control of the constitutive human phosphoglycerate kinase 1 (PGK) promoter with a SV40 polyA, is flanked by homology arms (250 bp each) and cloned in a standard AAV vector backbone (AAV2) in sense orientation with respect to its ITR (Inverted Terminal Repeats).

The lentiviral vector encoding the β^{AS3} gene under the control of the erythroid specific β-globin enhancer/promoter was already described (Weber et al., Mol. Ther. Methods Clin. Dev. 2018; 10:268-280)

All recombinant single stranded AAV2/6 used in this study are produced using a triple transfection protocol and purified by two sequential cesium chloride (CsCl) density gradients or chromatography as described in Ayuso E et al. Curr Gene Ther. 2010; 10(6):423-436. The vector titer of each preparation is determined by quantitative real-time PCR-based titration method using primers and probe corresponding to the ITR region of the AAV genome (Rohr U P et al. J Virol Methods. 2002; 106(1):81-88).

Lentiviruses (LV) are produced by transient transfection of 293T using third-generation packaging plasmid pMDLg/p.RRE and pK.REV and pseudotyped with the vesicular stomatitis virus glycoprotein G (VSV-G) envelope. LV are titrated in HCT116 cells and HIV-1 Gag p24 content is measured by ELISA (Perkin-Elmer) according to manufacturer's instructions.

After manipulation, HSPCs are cultured in erythroid differentiation medium (StemSpan, StemCell Technologies, Vancouver, BC, Canada; rhSCF 20 ng/ml, rhEpo 1 U/mL, IL3 5 ng/ml, Dexamethasone 2 μM and Bestradiol 1 μM; Sigma-Aldrich, St. Louis, Miss., USA) or in semisolid Methocult medium (colony-forming cells (CFC) assay, H4435, StemCell Technologies, Vancouver, BC, Canada) for 14 days. Colonies are counted and identified according to morphological criteria (BFU-E, CFU-G/GM, and CFU-GEMM). In some experiments, BFU-E are picked and cultured in erythroid progenitor expansion medium, as described in Wen J et al. J Hematol Oncol. 2017; 10(1):119.

48 hours after editing, 5-7×10⁵ CD34+ cells are injected intravenously into 8-week-old female NSG mice after sublethal irradiation (150cGy).

Human cell engraftment and KI levels are monitored at different time points in peripheral blood by flow cytometry using anti human CD45 (Fluorochrome APC, Clone HI30 from BD Bioscience; catalog Number 555485) and HLA-ABC antibodies (Fluorochrome PE, Clone G46-2.6 from BD Bioscience; catalog Number 555553). 16 weeks after transplantation, blood, bone marrow and spleen are harvested and analyzed. Peripheral blood is stained and red blood cells lysed during sample fixation (VersaLyse Lysing Solution and IOTest3 Fixative solution, Beckman Coulter, Pasadena, Calif., USA) using manufacturer's instructions.

Human CD34+ or CD45+ cells are purified from mouse peripheral blood or bone marrow by immunomagnetic selection with CD34 or CD45 MicroBead Kit UltraPure in combination with AUTOMACS PRO (PosselD2 separation program; Miltenyi Biotec, Paris, France) using manufacturer's instructions.

Statistical analyses are performed using GraphPad Prism version 6.00 for Windows (GraphPad Software, La Jolla, Calif., USA, www.graphpad.com). One-way or two-way analysis of variance (ANOVA) with Tukey's multiple comparison post-test for three or more groups is performed as indicated. Values are expressed as mean±standard deviation (SD) as otherwise indicated.

Results

The results obtained illustrate that HSPCs can be efficiently edited according to the invention and retain long-term and multilineage engraftment potential.

To evaluate this strategy in clinically relevant cells, and as detailed above, the inventors transfected human umbilical cord blood HSPCs with RNP and then transduced with AAV. Without any selection, the inventors achieved robust genome cutting (FIG. 7A), which resulted in efficient HBA2 deletion and donor DNA KI (FIGS. 7B et 7C).

Upon differentiation, HSPC-derived erythroblasts expressed β^{AS3}-mRNA, which accounted for about 15% (14.2±9.4, n=6) of total β globin RNA (FIG. 7D). The inventors then plated HSPCs in methylcellulose (CFC assay (colony-forming cells)) and confirmed that modified progenitors retained their multilineage potential (FIG. 8).

Single BFU-E (burst-forming unit-erythroid) genotyping showed that more than half of the colonies had HBA2 deletion (FIG. 9A) and β^{AS3} KI (FIG. 9B) and 41% had both modifications (see following Table):

| | wt | 1 β^{AS3} | 2 β^{AS3} | Tot |
|---|---|---|---|---|
| wt | 20 | 7 | 1 | 28 |
| 1 del | 6 | 17 | 2 | 25 |
| 2 del | 0 | 5 | 0 | 5 |
| Tot | 26 | 29 | 3 | |

Both HBA2 deleted and β^{AS3} KI HSPCs were then transplanted in immunodeficient NOD/SCID/γ (NSG) mice to evaluate their in vivo homing, engraftment and multilineage potential. Both edited HSPCs showed successful engraftment in bone marrow (BM), spleen (SP) and blood (PB) (FIG. 10A) and lineage differentiation (FIG. 10B—human B cells (B), human T cells (T) and human myeloid cells (My)).

Since NSG mice do not support human erythroid differentiation (Ishikawa F et al. Blood. 2005; 106(5):1565-1573), the inventors confirmed differentiation by CFC assay of isolated human CD34+ cells from bone marrow of engrafted mice (FIG. 11).

In addition, the percentage of InDels (FIG. 12A), as well as the extent of HBA2 deletion (FIG. 12B), remained similar in ex vivo and in vivo RNP treated HSPCs, confirming a similar efficiency in both stem and progenitor cells.

β^{AS3} KI HSPC (GFP positive) were present at different time points (FIG. 13A) in different lineages (FIG. 13B).

Overall, these data show that the inventors were able to achieve efficient HBA2 deletion and β^{AS3} KI in HSPCs while preserving their in vivo long-term engraftments well as their ability to differentiate.

### Example 7: Editing Patient's HSPCs Ameliorates β+- and β0-Thalassemia Phenotype

To test the invention in therapeutic conditions, the inventors assessed correction of globin imbalance in β-thalassemic patients' HSPCs.

In particular, the inventors tested the HBA2 deletion approach in β+ cells and the combination of the α-deletion and β^{AS3} KI strategy in β+- and β0-thalassemic HSPCs from actual patients.

The HSPCs are transfected with RNP and transduced with AAV as described above. As positive control, HSPCs are also transduced with a LV coding for a β^{AS3} transgene under the control of the β-globin gene promoter and its mini-locus control region (LCR), currently in clinical trial for thalassemia (LV β^{AS3}; Marktel S et al. Nat Med. 2019; 25(2):234-241.; Miccio A et al. Proc Natl Acad Sci USA. 2008; 105(30):10547-10552).

RNP transfection is very efficient both in erythroid liquid culture and CFC (Colony-forming cells) in generating InDels (90.5%±7.1 for RNP and 90.4%±9.0 for RNP+AAV, mean±SD; FIG. 14A), HBA2 deletion (0.95±0.06 HBA2 copies/cell for RNP and 0.77±0.15 for RNP+AAV, mean±SD; FIG. 14B) and β^{AS3} KI (0.80±0.21 on-target copies/cell for RNP+AAV, mean±SD; FIG. 14C).

Globin expression is monitored during erythroid differentiation.

mRNA globin imbalance (measured as a/β-like globins ratio) is ameliorated in all conditions (FIG. 14D), with β^{AS3} KI cells performing better than HBA2 deleted erythroblasts, due to β^{AS3} expression (FIGS. 14E et 14F). Noteworthy, modified HSPCs retained proper erythroid differentiation and multilineage potential. Globin mRNA analysis of single BFU-E colonies derived from β0 and β+ edited HSPCs showed that α/β imbalance is improved in all conditions, with the strongest effect due to the concomitant α downregulation and β^{AS3} expression (FIG. 14G).

Overall, these data show that the inventors are able to modify β-thalassemic HSPCs and reduce their α/β globin balance, without affecting HSPCs potential.

Targeted sequences Table

| Targeted | HBA gRNA | Partial sequence | SEQ ID | Full Sequence targeted | SEQ ID |
|---|---|---|---|---|---|
| Region | name | targeted by the gRNA | NO | by the gRNA | NO |
| 5′ | HBA 10 | GGGTTTATGCTTGGGGC | 1 | GGGTTTATGCTTGGGGCGCGG | 2 |
| region | | GCG | | GG | |
| | HBA 12 | GACTCAGAGAGAACCC | 3 | GACTCAGAGAGAACCCACCAT | 4 |
| | | ACCA | | GG | |
| | HBA 14 | TGGGTTCTCTCTGAGTC | 5 | TGGGTTCTCTCTGAGTCTGTGG | 6 |
| | | TGT | | G | |
| | HBA 15.1 | GGGTTCTCTCTGAGTCT | 7 | GGGTTCTCTCTGAGTCTGTGG | 8 |
| | | GTG | | GG | |
| | HBA 16.1 | GTCGGCAGGAGACAGC | 9 | GTCGGCAGGAGACAGCACCAT | 10 |
| | | ACCA | | GG | |
| | HBA 17 -G | GCAGGAGACAGCACCA | 11 | GCAGGAGACAGCACCATGGTG | 12 |
| | | TGGT | | GG | |
| | HBA 20.1 | CATAAACCCTGGCGCGC | 13 | CATAAACCCTGGCGCGCTCGC | 14 |
| | | TCG | | GG | |
| 3′ | HBA 5.1 | TTGAATGCTCCAGCCGG | 15 | TTGAATGCTCCAGCCGGTTCCA | 16 |
| UTR | | TTC | | G | |
| | gRNA2 | CGGGAGGCTTCGCCCA | 17 | CGGGAGGCTTCGCCCAATCCT | 18 |
| | | ATCC | | GG | |
| | gRNA3 | CGGGCGAGCGAGTGCG | 19 | CGGGCGAGCGAGTGCGAGCC | 20 |
| | | AGCC | | GG | |
| | gRNA 11 | GGGAGGCTTCGCCCAAT | 21 | GGGAGGCTTCGCCCAATCCTG | 22 |
| | | CCT | | GG | |
| IVS1 | HBA INT1 | CAGGCCACCCTCAACCG | 23 | CAGGCCACCCTCAACCGTCCTG | 24 |
| | 72.1 | TCC | | G | |
| | HBA INT1 | TCCGGGGCCAGGACGG | 25 | TCCGGGGCCAGGACGGTTGAG | 26 |
| | 73.2 | TTGA | | GG | |
| | HBA INT1 | GTCCGGGGCCAGGACG | 27 | GTCCGGGGCCAGGACGGTTGA | 28 |
| | 73b.1 | GTTG | | GG | |
| IVS2 | HBA INT2 | CCCTCGACCCAGATCGC | 29 | CCCTCGACCCAGATCGCTCCCG | 30 |
| | 13.2 | TCC | | G | |
| | HBA INT2 | GCGTGATCCTCTGCCCT | 31 | GCGTGATCCTCTGCCCTGAGA | 32 |
| | 74.1 | GAG | | GG | |

1. A genetically modified hematopoietic stem cell (HSC) comprising, in at least one α-globin gene comprised in the genome thereof, at least one transgene encoding a functional β-like globin protein, the at least one transgene being placed under the control of an endogenous sequence allowing the transcription and/or enhancing transcription, of the said at least one α-globin gene.

2. The genetically modified hematopoietic stem cell according to claim 1, wherein the at least one α-globin gene comprising the at least one transgene encoding a functional β-like globin protein does not express a gene sequence encoding a functional α-globin protein.

3. The genetically modified hematopoietic stem cell according to claim 1, wherein the at least one transgene encodes a functional β-like globin protein selected from the group consisting of an ε-globin, a G-γ-globin, a A-γ-globin, a δ-globin and a β-globin protein.

4. The genetically modified hematopoietic stem cell according to claim 1, wherein the at least one transgene encoding a functional β-like globin protein is comprised in the 5′ region, in the 3′ untranslated region (3′ UTR) and/or in an intron of the at least one α-globin gene.

5. The genetically modified hematopoietic stem cell according to claim 4, wherein the at least one transgene encoding a functional β-like globin protein is comprised in the 5′ untranslated region (5′UTR) and/or in the proximal promoter and/or in the second intron (IVS2) of the at least one α-globin gene.

6. The genetically modified hematopoietic stem cell according to claim 1, wherein the at least one α-globin gene is selected from the group consisting of the α1-globin gene and the α2-globin gene.

7. A blood cell originating from a genetically modified hematopoietic stem cell according to claim 1.

8. A pharmaceutical composition comprising at least one genetically modified hematopoietic stem cell according to claim 1 and/or at least one blood cell originating from the genetically modified hematopoietic stem cell, in a pharmaceutically acceptable medium.

9. A method for the ex vivo or in vitro preparation of a genetically modified hematopoietic stem cell according to claim 1, comprising at least the steps of:
(i) providing to a hematopoietic stem cell a site-directed genetic engineering system by:
(a) providing to the hematopoietic stem cell (1) at least one guide nucleic acid binding to a selected target site or (2) at least one guide peptide-containing endonuclease binding to the selected target site, the selected target site being located in an endogenous α-globin gene comprised in the genome of the hematopoietic stem cell;
(b) when the at least one guide nucleic acid has been provided at step (a) (1), further providing to the hematopoietic stem cell at least one endonuclease devoid of target site specificity; and
(c) further providing to the hematopoietic stem cell at least one transgene that encodes a functional β-like globin protein; and
(ii) culturing the genetically modified hematopoietic stem cell obtained at step (i) such that the at least one transgene encoding a functional β-like globin protein is introduced at the selected target site in the genome of the genetically modified hematopoietic stem cell and placed under the control of the endogenous α-globin gene, thereby allowing transcription and/or enhancing transcription of the endogenous α-globin gene.

10. The method according to claim 9, wherein the method comprises the steps of:
(i) providing to the hematopoietic stem cell a site-directed genetic engineering system by:
(a) providing to the hematopoietic stem cell at least one guide nucleic acid binding to the selected target site, the selected target site being located in an endogenous α-globin gene comprised in the genome of the hematopoietic stem cell;
(b) further providing to the hematopoietic stem cell at least one Clustered regularly interspaced short palindromic repeats (CRISPR) associated nuclease; and
(c) further providing to the hematopoietic stem cell at least one transgene that encodes a functional β-like globin protein;
and
(ii) culturing the hematopoietic stem cell obtained at the end of step (i) such that the at least one transgene is introduced at the selected target site in the genome of the hematopoietic stem cell.

11. The method according to claim 10, wherein the at least one Clustered regularly interspaced short palindromic repeats (CRISPR) associated nuclease is the CRISPR associated protein 9 (Cas9).

12. (canceled)

13. A method for the treatment of a β-hemoglobinopathy in a subject in need thereof, comprising administering to the subject a therapeutically effective amount of the genetically modified hematopoietic stem cell according to claim 1, a blood cell originating from the genetically modified hematopoietic stem cell, or a pharmaceutical composition comprising the genetically modified hematopoietic stem cell and/or the blood cell originating from the genetically modified hematopoietic stem cell.

14. An in vitro or ex vivo method for restoring the physiological level of production of hemoglobin in a cell, comprising at least the steps of:
(i) providing to the cell a site-directed genetic engineering system by:
(a) providing to the cell (1) at least one guide nucleic acid binding to a selected target site or (2) at least one guide peptide-containing endonuclease binding to the selected target site, the selected target site being located in an endogenous α-globin gene comprised in the genome of the cell;
(b) when the at least one guide nucleic acid or the at least one guide peptide-containing endonuclease has been provided at step (a) (1), further providing to the cell at least one endonuclease devoid of target site specificity; and
(c) further providing to the cell at least one transgene that encodes a functional β-like globin protein;
(ii) culturing the cell obtained at step (i) such that the at least one transgene encoding a functional β-like globin protein is introduced at the selected target site in the genome of the cell and placed under the control of the endogenous α-globin gene, thereby allowing transcription and/or enhancing transcription of the at least one α-globin gene; and
(iii) culturing the cell obtained at step (ii) such that the at least one transgene encoding a functional β-like globin protein that has been introduced in the genome of the cell is transcribed by the endogenous transcription system of the cell.

15. A kit for restoring in vitro or ex vivo the physiological level of production of hemoglobin in a cell, comprising:
(i) at least one guide nucleic acid binding to a selected target site or at least one guide peptide-containing endonuclease binding to the selected target site, the selected target site being located in an endogenous α-globin gene comprised in the genome of a hematopoietic stem cell (HSG);
(ii) at least one endonuclease devoid of target site specificity; and
(iii) at least one transgene encoding a functional β-like globin protein.

16. The method according to claim 13, wherein the β-hemoglobinopathy is β-thalassemia.
